(19) United States
(12) Patent Application Publication
(10) Pub. No.: US 2026/0315525 A1
(43) Pub. Date: Oct. 15, 2026

(54) METHOD OF TREATING CANCER CELLS USING F-BLOCK ELEMENT-SUBSTITUTED ZINC OXIDE NANOPARTICLES

(71) Applicant: IMAM ABDULRAHMAN BIN FAISAL UNIVERSITY, Dammam (SA)

(72) Inventors: Yassine SLIMANI, Dammam (SA); Firdos Alam KHAN, Dammam (SA)

(73) Assignee: IMAM ABDULRAHMAN BIN FAISAL UNIVERSITY, Dammam (SA)

(21) Appl. No.: 19/179,580

(22) Filed: Apr. 15, 2025

(51) Int. Cl. A61K 33/30; A61K 9/14; A61K 33/244; A61P 35/00

(52) U.S. Cl. CPC A61K 33/30; A61K 9/14; A61K 33/244; A61P 35/00

(57) ABSTRACT

A method of treating cancer cells includes contacting cancer cells with ternary metal oxide nanoparticles to induce cell death of the cancer cells. The ternary metal oxide nanoparticles include a zinc oxide matrix doped with terbium and erbium. The terbium is present in an amount of 0.5 to 10 atomic percent (at. %), and the erbium is present in an amount of 0.5 to 10 at. %. Atomic percent is based on the total atom count of the zinc, terbium, and erbium. The erbium replaces zinc within the zinc oxide matrix. The ternary metal oxide nanoparticles have an average particle size of 22 to 32 nm.

### STATEMENT REGARDING PRIOR DISCLOSURE BY THE INVENTORS

Aspects of the present disclosure are described in Slimani, Y. et al., “Synthesis, characterization, DFT calculations, and anticancer properties of VI-period and f-block elements-substituted zinc oxide nanopowders” published in Issue 151, Optical Materials, 2024, which is incorporated herein by reference in its entirety.

### BACKGROUND

### Technical Field

The present disclosure is directed to a method of treating cancer cells and, more particularly, relates to a method of treating cancer cells using f-block element-substituted zinc oxide nanoparticles.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present disclosure.

Nanomaterials with dimensions <100 nm display beneficial properties compared to their bulk counterparts. Their properties depend on the shape and size in which they appear. Among various nanomaterials, metal oxide nanoparticles have aroused interest in different fields and are used due to their physiochemical characteristics. Zinc oxide (ZnO) nanoparticles (NPs), belonging to the class of inorganic materials, have been used in various biomedical applications, including bio-sensing, targeted drug delivery, antibacterial destruction, cancer therapy, and the like, owing to their biodegradability, biocompatibility, low toxicity, and cost-effectiveness. ZnO NPs bear appealing anti-cancer potential as they are more destructive to cancer cells with a level of selectivity for cancer cells over healthy cells [Aljabali, A. A. A. et al., Synthesis, characterization, and assessment of anti-cancer potential of ZnO nanoparticles in an in vitro model of breast cancer, Molecules, 2022, 27, 1827]. ZnO NPs can serve as an alternative medicinal method for treating cervical carcinoma by suppressing damaged cells without cytotoxic impacts in healthy cells [Mohamed Asik, R. et al., Anticancer potential of zinc oxide nanoparticles against cervical carcinoma cells synthesized via biogenic route using aqueous extract of Gracilaria edulis, Materials Science & Engineering: C, 2019, 103, 109840-109840]. ZnO nanoparticles have also displayed anti-cancer properties against breast cancer mediated by apoptosis and enhanced creation of reactive oxygen species (ROS) [Tabrez, S. et al., Biosynthesis of ZnO NPs from pumpkin seeds' extract and elucidation of its anticancer potential against breast cancer, Nanotechnology Reviews, 2022, 11 2714-2725]. ZnO NPs have also exhibited promising anticancer activity with selectivity towards gastric cancer cells [Du, W. et al., Controlled synthesis of zinc oxide nanoparticles through flame spray pyrolysis and evaluation of their anticancer effects against gastric cancer cell, Arabian Journal of Chemistry, 2023, 16, 105192]. The wide band gap (~3.38 eV), high binding energy in exciton (60 meV), and good transparency at 25° C. differentiate ZnO from other semiconductors and support its use in optical devices, electronic devices, cosmetic products, gas sensors, catalysis, and the like [Kumari, P. et al., A brief review on transition metal ion doped ZnO nanoparticles and its optoelectronic applications, Materials Today: Proceedings, 2021, 43, 3297-3302].

In past years, electronic, magnetic, optical, surface, and conducting properties, as well as bioactivities of ZnO, were improved by doping the ZnO with various elements. Doping reinforces the use of ZnO as a base material for light-emitting devices, solar cells, gas sensors, magnetic semiconductors, photo-catalysts, field-effect transistors, and biological systems (drug delivery, bio-imaging, and the like) [Carofiglio, M. et al., Doped zinc oxide nanoparticles: synthesis, characterization and potential use in nanomedicine, Applied Sciences, 2020, 10, 5194; and Senthilkumar, G. et al., Enhancement of antibacterial and anticancer properties lanthanum insight into zinc oxide nanoparticles prepared via coprecipitation process, Inorganic Chemistry Communications, 2023, 155, 111081]. Appropriate dopant choice (concentration and properties) in ZnO affect the implementation of the material in various fields. Among various dopants, lanthanide elements and rare earth (RE) ions attract special attention as they are widely used for diverse applications owing to their chemical, optical, and magnetic characteristics and their electronic structure with a partially filled f-orbital [Pawade, V. B. et al., Introduction to electronic spectroscopy of lanthanide, properties, and their applications, Spectroscopy of Lanthanide Doped Oxide Materials, pp. 1-20, Woodhead Publishing, 2020]. Nanostructures of ZnO are appropriate host materials for RE element dopants. Specifically, RE-doped ZnO shows promising results for biomedical purposes. Doping ZnO NPs with neodymium (Nd) changes their properties and enhances cytotoxicity and antibacterial effects [Indumathi, T. et al., Effects of Nd doping on structural, optical, morphological and surface-chemical state analysis of ZnO nanoparticles for antimicrobial and anticancer activities, Surfaces and Interfaces, 2021, 23, 101000]. Improved antibacterial and anticancer potentials have been reported for ZnO NPs doped with lanthanum (La) [Senthilkumar, G. et al., Enhancement of antibacterial and anticancer properties lanthanum insight into zinc oxide nanoparticles prepared via coprecipitation process, Inorganic Chemistry Communications, 2023, 155, 111081]. Samarium (Sm) doped ZnO NPs may be considered as a new antitumor candidate with high efficiency and low toxicity [Nabeel, A. I., Samarium enriches antitumor activity of ZnO nanoparticles via downregulation of CXCR4 receptor and cytochrome P450, Tumor Biology, 2020, 42, 3]. RE ions can be good doping agents in ZnO NPs for photocatalytic applications, light-emitting devices, and solar cells [Rezaie, M. N. et al., Hybrid inorganic-organic light-emitting heterostructure devices based on ZnO, Optics & Laser Technology, 2021, 138, 106896].

As RE elements, terbium (Tb) and erbium (Er) can effectively enhance properties of ZnO. Erbium usually exists in its trivalent state (Er³⁺, 4f¹¹). Erbium may be utilized as a doped modifier in zinc oxide owing to its intra-4f shell transitions that may engender photoemission at 1.54 μm [Ianhez-Pereira, C. et al., Tailoring Stark effect in the 1.54 μm emission of Er-doped ZnO thin films, Scripta Materialia, 2021, 192, 102-105] and efficient modulation of deep-level emissions [Sun, S-Y. et al., Synthesis of Er³⁺ doped KNbO₃ nanocrystals and nanoceramics with outstanding up-conversion luminescence behaviors, Journal of Alloys and Compounds, 2021, 854, 156738]. Er-doped ZnO synthesized by a conventional ceramic method showed an enhanced visible emission associated with the creation of defects caused by the incorporation of Er in the ZnO lattice [Divya, N. K. and Pradyumnan, P. P., Solid state synthesis of erbium-doped ZnO with excellent photocatalytic activity and enhanced visible light emission, Materials Science in Semiconductor Processing, 2016, 41, 428-435]. ZnO nanorods doped with 0.5% Er can be used as an efficient photocatalyst for solar-driven environmental remediation [Choudhary, S. and Mohapatra, S., Enhanced photocatalytic activity of Er-doped ZnO nanospindles and nanorods for degradation of organic pollutants, Inorganic Chemistry Communications, 2024, 161, 111977].

Like erbium, terbium (Tb) is usually stable in the +3-oxidation state. Tb³⁺ has a wide absorption and bright green emission. These properties make it operative in designing advanced electronic devices such as light-emitting diodes (LEDs), fluorescent lamps, plasma display panels (PDPs), cathode ray tubes, plasma screens, liquid-crystal displays (LCDs), and the like [Lompré, J. S. et al., Bioaccumulation and ecotoxicological responses of clams exposed to terbium and carbon nanotubes: Comparison between native (Ruditapes decussatus) and invasive (Ruditapes philippinarum) species, Science of The Total Environment, 2021, 784, 146914]. Terbium has displayed a suitability as a dopant element in a host matrix in gas sensing applications because of its stability in air. For example, incorporating 4% terbium into ZnO achieves the highest sensing response towards acetone and ethanol [Hastir, A. et al., Temperature-dependent selective and sensitive terbium doped ZnO nanostructures, Sensors and Actuators B: Chemical, 2016, 231, 110-119]. ZnO—Tb³⁺ films showed a strong green luminescence in the spectral region of about 1.9 to 2.6 eV [Malyutina-Bronskaya, V. et al., Structural, optical and photoelectric properties of Tb doped ZnO thin films for device applications, Optical Materials, 2022, 127, 112305].

Although there are many experimental investigations on pure ZnO and mono-RE element-doped zinc oxide, investigations on the features of ZnO doped simultaneously by two types of RE elements or more are limited. Accordingly, an object of the present disclosure is co-doping ZnO with Tb and Er ions to change properties and evaluate the effects of Tb and Er co-doped ZnO nanoparticles on cancer cells to overcome limitations, such as toxicity and selectivity, of the art.

### SUMMARY

In an exemplary embodiment, a method of treating cancer cells is described. The method includes contacting cancer cells with ternary metal oxide nanoparticles to induce cell death of the cancer cells. The ternary metal oxide nanoparticles comprise a zinc oxide matrix doped with terbium and erbium. The terbium is present in an amount of 0.5 to 10 atomic percent (at. %) and the erbium is present in an amount of 0.5 to 10 at. %, where the atomic percent is based on a total atom count of the zinc, terbium, and erbium. The erbium replaces zinc within the zinc oxide matrix. The ternary metal oxide nanoparticles have an average particle size of 22 to 32 nm.

In some embodiments, the terbium is on a surface of the zinc oxide matrix.

In some embodiments, the ternary metal oxide nanoparticles do not contain an erbium oxide phase.

In some embodiments, the ternary metal oxide nanoparticles comprise a terbium oxide phase.

In some embodiments, the ternary metal oxide nanoparticles have a lattice parameter (a) of 0.32 to 0.33 nm.

In some embodiments, the ternary metal oxide nanoparticles have a lattice parameter (c) of 0.51 to 0.53 nm.

In some embodiments, the ternary metal oxide nanoparticles have a band gap energy of 1.5 to 3 eV.

In some embodiments, the ternary metal oxide nanoparticles have a crystallite size of 10 to 25 nm.

In some embodiments, the ternary metal oxide nanoparticles comprise oxygen in an amount of 50 to 60 at. %, zinc in an amount of 35 to 45 at. %, terbium in an amount of 0.5 to 5 at. %, and erbium in an amount of 0.5 to 5 at. % based on a total atom count of the oxygen, zinc, terbium, and erbium.

In some embodiments, the ternary metal oxide nanoparticles have a peak in an extinction spectrum at 0.1 to 1 eV corresponding to an infrared region.

In some embodiments, the ternary metal oxide nanoparticles have a peak in an energy loss spectrum at 1 to 3 eV.

In some embodiments, the method of contacting cancer cells with ternary metal oxide nanoparticles occurs for 36 to 60 hours.

In some embodiments, the cancer cells are HCT-116 cells, and the HCT-116 cells have an average cell viability of 44 to 48% after 45 to 50 hours.

In some embodiments, the cancer cells are HeLa cells, and the HeLa cells have an average cell viability of 50 to 66% after 45 to 50 hours.

In some embodiments, a concentration of the ternary metal oxide nanoparticles is 30 to 50 μg/mL.

In some embodiments, apoptotic bodies are formed to induce cell death of the cancer cells.

In some embodiments, chromatin condensation occurs to induce cell death of the cancer cells.

In some embodiments, a process of making the ternary metal oxide nanoparticles is described. The process includes dissolving a zinc salt in a polar protic solvent to form a zinc solution, dissolving a terbium salt in a polar protic solvent to form a terbium solution, dissolving an erbium salt in a polar protic solvent to form an erbium solution, mixing and heating the zinc solution, the terbium solution, and the erbium solution to form a reaction solution, adding an acid to the reaction solution, adjusting the pH of the reaction solution to 6 to 8 with a base, heating the reaction solution to 200 to 400° C. to form a powder, and calcinating the powder for 3 to 5 hours at 400 to 600° C. to form the ternary metal oxide nanoparticles.

In some embodiments, the ternary metal oxide nanoparticles comprise terbium in an amount of 0.9 to 5.5 at. % based on a total atom count of the zinc, terbium, and erbium.

In some embodiments, the ternary metal oxide nanoparticles comprise erbium in an amount of 0.9 to 5.5 at. % based on a total atom count of the zinc, terbium, and erbium.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A is a flow chart depicting a process of making ternary metal oxide nanoparticles, according to certain embodiments.

FIG. 1B is an exemplary flow chart depicting a procedure of preparing terbium (Tb)/erbium (Er) co-doped zinc oxide (ZnO) nanopowders, according to certain embodiments.

FIG. 2A illustrates a supercell representing a zinc erbium terbium oxide (ZnErTbO-II) structure, according to certain embodiments.

FIG. 2B depicts X-ray diffraction (XRD) patterns of ZnO, ZnErTbO-I, ZnErTbO-II, and ZnErTbO-III, according to certain embodiments.

FIG. 2C depicts relation variations of lattice parameters (a and c) for ZnO, ZnErTbO-I, ZnErTbO-II, and ZnErTbO-III, according to certain embodiments.

FIG. 2D depicts relative variation (RV) in lattice parameters Δa/a₀ and Δc/c₀ for ZnO, ZnErTbO-I, ZnErTbO-II, and ZnErTbO-III, according to certain embodiments.

FIG. 2E depicts variations of degree of distortion “R” and c/a ratio for ZnO, ZnErTbO-I, ZnErTbO-II, and ZnErTbO-III, according to certain embodiments.

FIG. 3 depicts Williamson-Hall plots for ZnO, ZnErTbO-I, ZnErTbO-II, and ZnErTbO-III, according to certain embodiments.

FIG. 4A depicts a transmission electron microscopy (TEM) image for pure ZnO, according to certain embodiments.

FIG. 4B depicts a TEM image for ZnErTbO-II, according to certain embodiments.

FIG. 4C depicts a particle size distribution histogram for pure ZnO, according to certain embodiments.

FIG. 4D a depicts particle size distribution histogram for ZnErTbO-II, according to certain embodiments.

FIG. 5A depicts an energy-dispersive X-ray (EDX) spectra and a chemical composition for pure ZnO sample, according to certain embodiments.

FIG. 5B depicts a EDX spectra and a chemical composition for ZnErTbO-II, according to certain embodiments.

FIG. 6A depicts total density of states (TDOS) and partial density of states (PDOS) spectra for pure ZnO, according to certain embodiments.

FIG. 6B depicts TDOS and PDOS spectra for ZnErTbO-I, according to certain embodiments.

FIG. 6C depicts TDOS and PDOS spectra for ZnErTbO-II, according to certain embodiments.

FIG. 6D depicts TDOS and PDOS spectra for ZnErTbO-III, according to certain embodiments.

FIG. 7 depicts absorption coefficients as a function of energy for ZnO, ZnErTbO-I, ZnErTbO-II, and ZnErTbO-III, according to certain embodiments.

FIG. 8 depicts a refractive index for ZnO, ZnErTbO-I, ZnErTbO-II, and ZnErTbO-III, according to certain embodiments.

FIG. 9 depicts an imaginary part of a dielectric constant as a function of energy for ZnO, ZnErTbO-I, ZnErTbO-II, and ZnErTbO-III, according to certain embodiments.

FIG. 10 depicts a reflectivity of ZnO, ZnErTbO-I, ZnErTbO-II, and ZnErTbO-III, according to certain embodiments.

FIG. 11A depicts an extinction function of ZnO, ZnErTbO-I, ZnErTbO-II, and ZnErTbO-III, according to certain embodiments.

FIG. 11B depicts an energy loss function of ZnO, ZnErTbO-I, ZnErTbO-II, and ZnErTbO-III, according to certain embodiments.

FIG. 12 is a bar graph depicting cell viability assay results of a 3-(4,5-dimethylthiazol-2-yl)-2,5-diphenyltetrazolium bromide (MTT) assay showing the influence of ZnO, ZnErTbO-I, ZnErTbO-II, and ZnErTbO-III on HCT-116, HeLa, and HEK-293 cells, according to certain embodiments.

FIG. 13A depicts control cells (HCT-116) with normal morphology and cells that are intact and healthy, according to certain embodiments.

FIG. 13B depicts HCT-116 cells after 48 hours of treatment (40 μg/mL) with ZnO, according to certain embodiments.

FIG. 13C depicts HCT-116 cells after 48 hours of treatment (40 μg/mL) with ZnErTbO-I, according to certain embodiments.

FIG. 13D depicts HCT-116 cells after 48 hours of treatment (40 μg/mL) with ZnErTbO-II, according to certain embodiments.

FIG. 13E depicts HCT-116 cells after 48 hours of treatment (40 μg/mL) with ZnErTbO-III, according to certain embodiments.

### DETAILED DESCRIPTION

In the drawings, like reference numerals will be used to designate identical or corresponding parts throughout the several views. Further, as used herein, the words “a,” “an,” and the like generally carry a meaning of “one or more,” unless stated otherwise.

Furthermore, the terms “approximately,” “approximate”, “about,” and similar terms generally refer to ranges that include the identified value within a margin of 20%, 10%, or preferably 5%, and any values therebetween.

When describing the present disclosure, the terms used are to be construed in accordance with the following definitions, unless a context dictates otherwise.

Embodiments of the present disclosure will now be described more fully hereinafter with reference to the accompanying drawings wherever applicable, in that some, but not all, embodiments of the disclosure are shown.

When amounts, concentrations, dimensions, and other parameters are expressed in the form of a range, a preferable range, an upper limit value, a lower limit value, or preferable upper and limit values, it should be understood that any ranges obtainable by combining any upper limit or preferable value with any lower limit or preferable value are also specifically disclosed, irrespective of whether the obtained ranges are clearly mentioned in the context.

In this application, a numerical value interval (i.e., a numerical value range) is involved, and, if not specifically stated, an optional numerical value distribution is considered continuous within the numerical value interval and includes two numerical value endpoints (i.e., minimum and maximum values) of the numerical value range and each numerical value between the two numerical value endpoints.

The temperature parameters in the present application, if not specifically limited, are both allowed to be constant temperature processing and allowed to be varied within a certain temperature interval. The constant temperature processing allows the temperature to fluctuate within the precision range of the instrument control. It is allowed to fluctuate in the range of, for example, ±5° C., ±4° C., ±3° C., ±2° C., and ±1° C.

A weight percent of a component, unless specifically stated to the contrary, is based on the total weight of the formulation or composition in which the component is included. For example, if a particular element or component in a composition or article is said to have 5 weight percent (wt. %), it is understood that this percentage is in relation to a total compositional percentage of 100%.

The present disclosure is intended to include all hydration states of a given compound or formula, unless otherwise noted or when heating a material.

In addition, the present disclosure is intended to include all isotopes of atoms occurring in the present compounds and complexes. Isotopes include those atoms having the same atomic number but different mass numbers. By way of general example, and without limitation, isotopes of hydrogen include deuterium and tritium, and isotopes of carbon include ¹³C and ¹⁴C. Isotopes of oxygen include ¹⁶O, ¹⁷O, and ¹⁸O. Isotopically-labeled compounds of the disclosure may generally be prepared by conventional techniques known to those skilled in the art or by processes analogous to those described herein, using an appropriate isotopically-labeled reagent in place of the non-labeled reagent otherwise employed.

Aspects of the present disclosure are directed to a method of treating cancer cells with ternary metal oxide nanoparticles including ZnO co-doped with rare earth elements, Tb and Er, and preparing the ternary metal oxide nanoparticles via a sol-gel auto-combustion route. The anticancer properties of the ternary metal oxide nanoparticles were examined on cervical cancer (HeLa cells) and colon cancer (HCT-116) using an in vitro MTT assay. The assay results indicate a reduction in HeLa and HCT-116 cells after 48 hours of treatment of the ternary metal oxide nanoparticles. Treatment with co-doped ZnO nanoparticles produces chromatin condensation and forms apoptotic bodies in HCT-116 cells, which indicate that the co-doped ZnO nanoparticles induce programmed cell death in HCT-116 cells. The prepared Tb/Er co-doped ZnO nanoparticles have applications in treating cervical and colon cancers.

Aspects of the present disclosure are directed to a method of treating cancer cells. The cancer cells may be cancer of the thyroid, endocrine system, brain, breast, cervix, colon, head & neck, liver, kidney, lung, ovary, pancreas, rectum, stomach, uterus, a combination thereof, and the like. Additional examples may include, but are not limited to, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, prostate cancer, a combination thereof, and the like.

The method of treating cancer cells comprises contacting cancer cells with ternary metal oxide nanoparticles to induce cell death of the cancer cells. As used here, the term “ternary metal oxide” refers to a compound formed from three different metal elements and oxygen. The ternary metal oxide nanoparticles include a zinc oxide (ZnO) matrix doped with terbium (Tb) and erbium (Er). ZnO is used as a host matrix, and Tb and Er are co-doping ions. In some other embodiments, other f-block elements selected from lanthanum (La), cerium (Ce), praseodymium (Pr), neodymium (Nd), promethium (Pm), samarium (Sm), europium (Eu), gadolinium (Gd), dysprosium (Dy), holmium (Ho), thulium (Tm), ytterbium (Yb), lutetium (Lu), actinium (Ac), thorium (Th), protactinium (Pa), uranium (U), neptunium (Np) plutonium (Pu), americium (Am), curium (Cm), berkelium (Bk), californium (Cf), einsteinium (Es), fermium (Fm), mendelevium (Md), nobelium (No), lawrencium (Lr), and a combination there of may also be used with Tb and Er as co-doping ions.

The terbium is present in an amount of 0.5 to 10 atomic percent (at. %), preferably 1 to 9 at. %, preferably 2 to 8 at. %, preferably 3 to 7 at. %, and preferably 4 to 6 at. %, where atomic percent is based on a total atom count of zinc, terbium, and erbium. In some embodiments, the ternary metal oxide nanoparticles comprise terbium in an amount of 0.9 to 5.5 at. %, preferably 1 to 5 at. %, preferably 1.5 to 4 at. %, and preferably 2 to 3 at. % based on the total atom count of zinc, terbium, and erbium. The erbium is present in an amount of 0.5 to 10 atomic at. %, preferably 1 to 9 at. %, preferably 2 to 8 at. %, preferably 3 to 7 at. %, and preferably 4 to 6 at. %, where atomic percent is based on a total atom count of zinc, terbium, and erbium. In some embodiments, the ternary metal oxide nanoparticles comprise erbium in an amount of 0.9 to 5.5 at. %, preferably 1 to 5 at. %, preferably 1.5 to 4 at. %, and preferably 2 to 3 at. % based on the total atom count of zinc, terbium, and erbium. The erbium replaces zinc within the zinc oxide matrix. In some embodiments, the erbium replaces zinc on an inside of the zinc oxide matrix (for example, an inner vertex) within the zinc oxide matrix. In other embodiments, the erbium replaces zinc on an outer edge of the zinc oxide matrix that is within the zinc oxide matrix. In some other embodiments, the erbium replaces zinc on an inside of the zinc oxide matrix within the zinc oxide matrix and the erbium replaces zinc on an outer edge of the zinc oxide matrix that is within the zinc oxide matrix. In some embodiments, the erbium is on a surface of the zinc oxide matrix. In some embodiments, the erbium replaces zinc within the zinc oxide matrix and the erbium is on a surface of the zinc oxide matrix.

The ternary metal oxide nanoparticles have an average particle size of 22 to 32 nm, preferably 23 to 31 nm, preferably 24 to 30 nm, preferably 25 to 29 nm, more preferably 26 to 28 nm, and yet more preferably 26.5 to 27 nm. As used herein, the term “average particle size” refers to the mean diameter of nanoparticles in a sample, typically calculated from measurements obtained using techniques such as scanning electron microscopy (SEM) or transmission electron microscopy (TEM), representing the typical size of the particles in the distribution. In a specific embodiment, the average particle size of the ternary metal oxide nanoparticles is about 26.58 nm.

In some embodiments, X-ray diffractogram (XRD) patterns show that the ternary metal oxide nanoparticles have a wurtzite hexagonal structure. XRD technique further confirms the structural incorporation of Zn, Tb, and Er in the ternary metal oxide nanoparticles. In some embodiments, the terbium is present on the surface of the ZnO matrix, while erbium is incorporated into the ZnO lattice/matrix. In some embodiments, the terbium is on the surface of the zinc oxide matrix. This suggests that terbium is surface-bound and not incorporated into the ZnO lattice. In some embodiments, a fraction (for example, less than 5%, preferably less than 4%, preferably less than 3%, preferably less than 2%, preferably less than 1%, preferably less than 0.5%, and preferably less than 0.1%) of terbium ions may occupy Zn²⁺ sublattices and/or interstitial sites of the lattice; however, most of the terbium aggregates on the ZnO surface in the form of terbium oxide phase, preferably a Tb₂O₃ phase, a Tb₄O₇ phase, or a combination there. In some embodiments, the terbium replaces the zinc on an outer edge of the zinc oxide matrix that is within the zinc oxide matrix. In some embodiments, the ternary metal oxide nanoparticles comprise a terbium oxide phase, preferably a Tb₂O₃ phase. In some embodiments, the ternary metal oxide nanoparticles do not contain an erbium oxide phase. In some embodiments, the ternary metal oxide nanoparticles contain less than 5%, preferably less than 4%, preferably less than 3%, preferably less than 2%, preferably less than 1%, more preferably less than 0.5%, and yet more preferably less than 0.1% erbium oxide (Er₂O₃) phase based in the XRD spectrum.

In some embodiments, the ternary metal oxide nanoparticles have a lattice parameter (a) of 0.32 to 0.33 nm, preferably 0.321 to 0.329 nm, preferably 0.322 to 0.328 nm, preferably 0.323 to 0.327 nm, and preferably 0.324 to 0.326 nm. In some embodiments, the ternary metal oxide nanoparticles have a lattice parameter (c) of 0.51 to 0.53 nm, preferably 0.512 to 0.528 nm, preferably 0.514 to 0.526 nm, preferably 0.516 to 0.524 nm, and preferably 0.518 to 0.522 nm. In some embodiments, the ternary metal oxide nanoparticles have a c/a ratio of about 1.6, preferably in the range of 1.6 to 1.605, preferably 1.6005 to 1.604, and preferably 1.601 to 1.603.

In some embodiments, the ternary metal oxide nanoparticles have a crystallite size of 10 to 25 nm, preferably 12 to 23 nm, preferably 14 to 21 nm, preferably 16 to 19 nm, and preferably 17 to 18 nm. In a specific embodiment, the ternary metal oxide nanoparticles have a crystallite size of about 12.85 nm. In another specific embodiment, the ternary metal oxide nanoparticles have a crystallite size of about 19.13 nm. In another specific embodiment, the ternary metal oxide nanoparticles have a crystallite size of about 22.95 nm.

In some embodiments, the ternary metal oxide nanoparticles comprise oxygen in an amount of 50 to 60 at. %, preferably 52 to 59 at. %, more preferably 54 to 58 at. %, and yet more preferably 56 to 57 at. %, zinc in an amount of 35 to 45 at. %, preferably 36 to 44 at. %, preferably 37 to 43 at. %, preferably 38 to 42 at. %, more preferably 39 to 41.5 at. %, and yet more preferably 40 to 41 at. %, terbium in an amount of 0.5 to 5 at. %, 0.6 to 4 at. %, preferably 0.8 to 3 at. %, more preferably 1 to 2 at. %, and yet more preferably 1.2 to 1.5 at. %, and erbium in an amount of 0.5 to 5 at. %, preferably 0.6 to 4 at. %, preferably 0.8 to 3 at. %, more preferably 1 to 2 at. %, and yet more preferably 1.2 to 1.5 at. % based on a total atom count of the oxygen, zinc, terbium, and erbium. In a specific embodiment, the ternary metal oxide nanoparticles include oxygen in an amount of about 56.44 at. %, zinc in an amount of about 40.97 at. %, terbium in an amount of about 1.33 at. %, and erbium in an amount of about 1.27 at. % based on a total atom count of the oxygen, zinc, terbium, and erbium.

In some embodiments, the ternary metal oxide nanoparticles have a band gap energy of 1.5 to 3 eV, preferably 1.8 to 2.9 eV, preferably 2 to 2.6 eV, and preferably 2.2 to 2.4 eV. In a specific embodiment, the ternary metal oxide nanoparticles have a band gap energy of about 2.89 eV. In another specific embodiment, the ternary metal oxide nanoparticles have a band gap energy of about 2.26 eV. In another specific embodiment, the ternary metal oxide nanoparticles have a band gap energy of about 1.97 eV.

In some embodiments, the ternary metal oxide nanoparticles have a peak in an extinction spectrum at 0.1 to 1 eV, preferably 0.2 to 0.9 eV, preferably 0.3 to 0.8 eV, preferably 0.4 to 0.7 eV, and preferably 0.5 to 0.6 eV, corresponding to an infrared region. This is lower compared to pure ZnO, which exhibits a corresponding peak in the UV region at about 5 eV. In a specific embodiment, the ternary metal oxide nanoparticles have a peak in an extinction spectrum at about 0.4 eV. In some embodiments, the ternary metal oxide nanoparticles have a peak in an energy loss spectrum at 1 to 3 eV, preferably at about 1.2 to 2.8 eV, preferably 1.4 to 2.6 eV, preferably 1.6 to 2.4 eV, and preferably 1.8 to 2.2 eV.

The method of treating cancer includes contacting cancer cells with ternary metal oxide nanoparticles to induce cell death of the cancer cells. In some embodiments, a concentration of the ternary metal oxide nanoparticles is sufficient to induce cancer cell death. In some embodiments, a concentration of the ternary metal oxide nanoparticles is 30 to 50 μg/mL, preferably 32 to 48 μg/mL, preferably 35 to 45 μg/mL, preferably 37 to 43 μg/mL, more preferably 39 to 41 μg/mL, and yet more preferably about 40 μg/mL. In some embodiments, the method of contacting the cancer cells with the ternary metal oxide nanoparticles occurs for 36 to 60 hours, preferably 38 to 58 hours, preferably 40 to 56 hours, preferably 42 to 54 hours, preferably 44 to 52 hours, more preferably 46 to 50 hours, and yet more preferably about 48 hours.

In some embodiments, contacting the cancer cells with the ternary metal oxide nanoparticles forms apoptotic bodies to induce cell death of the cancer cells. As used herein, the term “apoptotic bodies” refers to extracellular vesicles (EVs) released during programmed cell death (i.e., apoptosis). Apoptotic bodies are released by dying cells and contain bioactive matter and information for intercellular communications. In some embodiments, contacting the cancer cells with the ternary metal oxide nanoparticles results in chromatin condensation to induce cell death of the cancer cells. As used herein, “chromatin condensation” refers to a process of compacting and reorganizing the structure of DNA during cell division. In some embodiments, cell shrinkage, chromatin condensation, plasma membrane blebbing without rupture, formation of apoptotic bodies, and cytoskeletal disintegration may be used and/or observed to induce death of the cancer cells.

In some embodiments, the cancer cells are HCT-116 cells, and the HCT-116 cells have an average cell viability of 44 to 48%, preferably 44.5 to 47.5%, preferably 45 to 47%, and preferably 45.5 to 46.5%, after contacting the HCT-116 cells with the ternary metal oxide nanoparticles for 45 to 50 hours, preferably 46 to 49.5 hours, and more preferably 47 to 49 hours. In one specific embodiment, the cancer cells are HCT-116 cells, and the HCT-116 cells have an average cell viability of about 44.83% after contacting the HCT-116 cells with the ternary metal oxide nanoparticles for about 48 hours. In another specific embodiment, the cancer cells are HCT-116 cells, and the HCT-116 cells have an average cell viability of about 46.24% after contacting the HCT-116 cells with the ternary metal oxide nanoparticles for about 48 hours. In yet another specific embodiment, the cancer cells are HCT-116 cells, and the HCT-116 cells have an average cell viability of about 47.26% after contacting the HCT-116 cells with the ternary metal oxide nanoparticles for about 48 hours.

In some embodiments, the cancer cells are HeLa cells, and the HeLa cells have an average cell viability of 50 to 66%, preferably 51 to 65%, preferably 52 to 64%, preferably 53 to 63%, preferably 54 to 62%, preferably 55 to 61%, preferably 56 to 60%, and preferably 57 to 59% after contacting the HeLa cells with the ternary metal oxide nanoparticles for 45 to 50 hours, preferably 46 to 49.5 hours, and more preferably 47 to 49 hours. In one specific embodiment, the cancer cells are HeLa cells, and the HeLa cells have an average cell viability of about 65.04% after contacting the HeLa cells with the ternary metal oxide nanoparticles for about 48 hours. In another specific embodiment, the cancer cells are HeLa cells, and the HeLa cells have an average cell viability of about 50.77% after contacting the HeLa cells with the ternary metal oxide nanoparticles for about 48 hours. In yet another specific embodiment, the cancer cells are HeLa cells, and the HeLa cells have an average cell viability of about 63.41% after contacting the HeLa cells with the ternary metal oxide nanoparticles for about 48 hours.

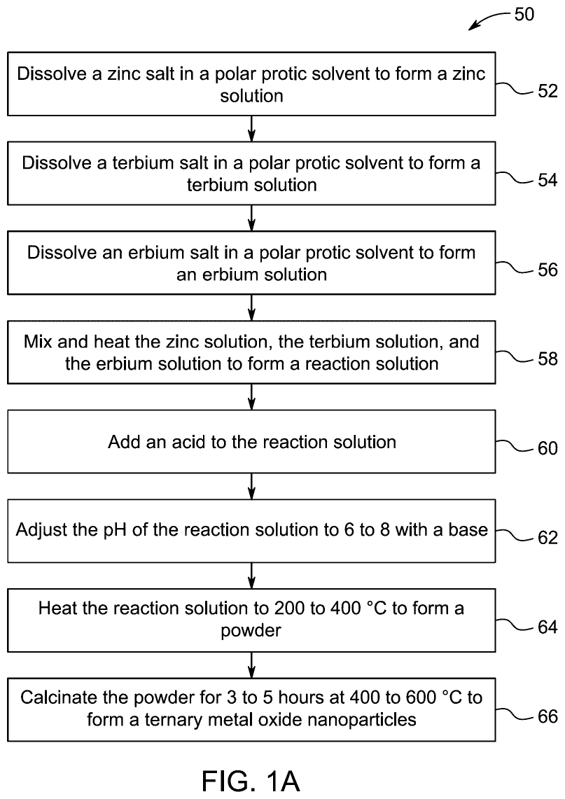

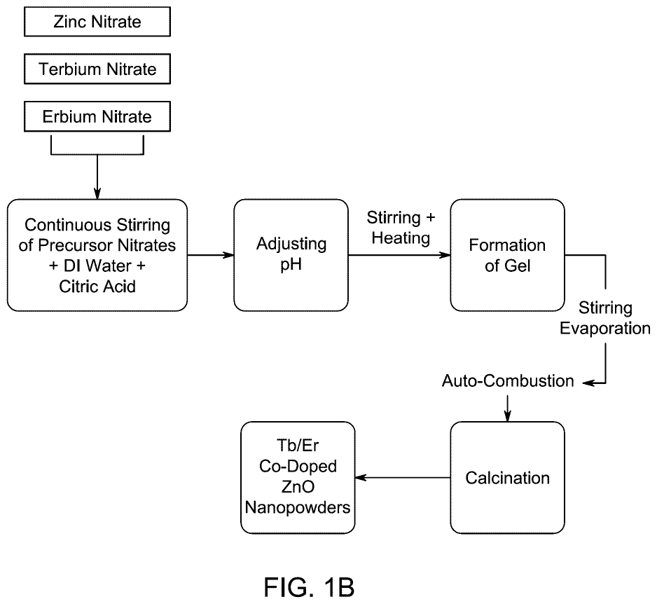

FIG. 1A refers to a method 50 of making ternary metal oxide nanoparticles. The order in which the method 50 is described is not intended to be construed as a limitation, and any number of the described method steps may be combined in any order to implement the method 50. Additionally, individual steps may be removed or skipped from the method 50 without departing from the spirit and scope of the present disclosure.

At step 52, the method 50 includes dissolving a zinc salt in a polar protic solvent to form a zinc solution. Examples of the zinc salt may include, but are not limited to, zinc chloride, zinc acetate, zinc sulfate, zinc carbonate, zinc nitrate, zinc bromide, zinc iodide, zinc sulfate heptahydrate, zinc chloride hexahydrate, zinc acetate dihydrate, zinc nitrate tetrahydrate, zinc thiocyanate, zinc formate, zinc methanesulfonate, zinc isothiocyanate, zinc citrate, zinc pyrophosphate, zinc orthophosphate, mixtures thereof, and the like. In a preferred embodiment, the zinc salt is Zn(NO₃)₂·6H₂O. The zinc salt is dissolved in the polar protic solvent to form the zinc solution. In some embodiments, the polar protic solvent may be, but is not limited to, water, methanol, ethanol, n-propanol, t-butanol, acetic acid, mixtures thereof, and the like. In a preferred embodiment, the polar protic solvent is water. The water may be tap water, distilled water, double-distilled water, deionized water, deionized distilled water, reverse osmosis water, and/or some other water.

At step 54, the method 50 includes dissolving a terbium salt in a polar protic solvent to form a terbium solution. Suitable examples of terbium salt include, but are not limited to, terbium(III) nitrate (Tb(NO₃)₃), terbium(III) chloride (TbCl₃), terbium(III) sulfate (Tb₂(SO₄)₃), terbium(III) acetate (Tb(C₂H₃O₂)₃), terbium(III) fluoride (TbF₃), terbium(III) carbonate (Tb₂(CO₃)₃), terbium(III) oxalate (Tb₂(C₂O₄)₃), mixtures thereof, and the like. In a preferred embodiment, the terbium salt is terbium nitrate. The terbium salt is dissolved in the polar protic solvent to form the terbium solution. In some embodiments, the polar protic solvent may be, but is not limited to, water, methanol, ethanol, n-propanol, t-butanol, acetic acid, mixtures thereof, and the like. In a preferred embodiment, the polar protic solvent is water.

At step 56, the method 50 includes dissolving an erbium salt in a polar protic solvent to form an erbium solution. Suitable examples of the erbium salt include, but are not limited to, erbium(III) nitrate (Er(NO₃)₃), erbium(III) chloride (ErCl₃), erbium(III) sulfate (Er₂(SO₄)₃), erbium(III) acetate (Er(C₂H₃O₂)₃), erbium(III) fluoride (ErF₃), erbium(III) carbonate (Er₂(CO₃)₃), erbium(III) oxalate (Er₂(C₂O₄)₃), mixtures thereof, and the like. In a preferred embodiment, the erbium salt is erbium nitrate. The erbium salt is dissolved in the polar protic solvent to form the erbium solution. In some embodiments, the polar protic solvent may be, but is not limited to, water, methanol, ethanol, n-propanol, t-butanol, acetic acid, mixtures thereof, and the like. In a preferred embodiment, the polar protic solvent is water.

At step 58, the method 50 includes mixing and heating the zinc solution, the terbium solution, and the erbium solution to form a reaction solution. The zinc solution, the terbium solution, and the erbium solution are combined and heated to promote reaction and homogenization, forming a reaction solution. In a preferred embodiment, the zinc solution, the terbium solution, and the erbium solution are mixed and stirred for 10 to 30 minutes, preferably 12 to 25 minutes, and preferably 15 to 20 minutes. In a preferred embodiment, the zinc solution, the terbium solution, and the erbium solution is mixed and heated to a temperature of 60 to 100° C., preferably 65 to 95° C., preferably 70 to 90° C., more preferably 75 to 85° C., and preferably about 80° C.

At step 60, the method 50 includes adding an acid to the reaction solution. An acid including, but not limited to, citric acid, nitric acid, acetic acid, oxalic acid, tartaric acid, glycolic acid, a combination thereof, and the like is added to the reaction solution to assist in the auto-combustion reaction due its role as a fuel, complexing agent, and pH stabilizer. In a preferred embodiment, the acid is citric acid. Citric acid chelates (binds) with metal ions (Zn²⁺, Tb³⁺, Er³⁺) to form stable metal-citrate complexes. This promotes uniform mixing of metal ions and prevents precipitation.

At step 62, the method 50 includes adjusting the pH of the reaction solution to 6 to 8, preferably 6.1 to 7.9, preferably 6.2 to 7.8, preferably 6.3 to 7.7, preferably 6.4 to 7.6, preferably 6.5 to 7.5, preferably 6.6 to 7.4, preferably 6.7 to 7.3, preferably 6.8 to 7.2, more preferably 6.9 to 7.1, and yet more preferably about 7 with a base. This is performed to control precipitation and metal hydroxide formation. Suitable examples of bases include, but are not limited to, ammonia, ammonium hydroxide, sodium hydroxide, potassium hydroxide, sodium carbonate, sodium bicarbonate, ethanolamine, urea, a combination thereof, and the like. In a preferred embodiment, the base is ammonium hydroxide.

At step 64, the method 50 includes heating the reaction solution to 200 to 400° C., preferably 210 to 390° C., preferably 220 to 380° C., preferably 230 to 370° C., preferably 240 to 360° C., preferably 250 to 350° C., preferably 260 to 340° C., preferably 270 to 330° C., preferably 280 to 320° C., more preferably 290 to 310° C., and yet more preferably about 300° C., to form a powder. In some embodiments, the heating can be performed by using heating appliances such as ovens, microwaves, autoclaves, hot plates, heating mantles and tapes, oil baths, salt baths, sand baths, air baths, hot-tube furnaces, hot-air guns, a combination thereof, and the like.

At step 66, the method 50 includes calcinating the powder for 3 to 5 hours, preferably 3.5 to 4.5 hours, and more preferably about 4 hours at 400 to 600° C., preferably 420 to 580° C., preferably 440 to 560° C., preferably 460 to 540° C., more preferably 480 to 520° C., and yet more preferably about 500° C. to form the ternary metal oxide nanoparticles. The calcination of the powder is carried out by heating it to a high temperature, under a restricted supply of ambient oxygen. This is performed to remove impurities or volatile substances and to incur thermal decomposition. Typically, the calcination is carried out in a furnace, preferably equipped with a temperature control system, which may provide a heating rate of up to 50° C. per minute (° C./min), preferably up to 40° C./min, preferably up to 30° C./min, preferably up to 20° C./min, preferably up to 10° C./min, preferably up to 5° C./min, preferably up to 2° C./min, and preferably up to 1° C./min. In a preferred embodiment, the powder is calcined at 500° C. for 4 hours.

### EXAMPLES

The following examples describe and demonstrate a method for treating cancer cells using f-block element-substituted zinc oxide nanoparticles. The examples are provided solely for illustration and are not to be construed as limitations of the present disclosure, as many variations thereof are possible without departing from the spirit and scope of the present disclosure.

### Example 1: Chemicals and Instruments

The following reagents (purity ≥99.9%) were used for synthesis: zinc nitrate hexahydrate (Zn(NO₃)₂·6H₂O), erbium nitrate pentahydrate (Er(NO₃)₂·5H₂O), and terbium(III) nitrate pentahydrate (Tb(NO₃)₃·5H₂O) as starting precursors. Citric acid (C₆H₈O₇) was used as a fuel and ammonium hydroxide (NH₃H₂O) was used to adjust the solution's pH. A Rigaku MiniFlex 600 X-ray diffractometer with Cuka radiation and a FEI Morgagni 268 transmission electron microscope was used to examine the crystallographic structure, the morphology, and size distribution of nanoparticles/nanopowders. Elemental analysis was carried out using energy-dispersive X-ray (EDX) spectroscopy (AMETEK EDAX Element).

### Example 2: Preparation

Tb/Er co-doped ZnO nanopowders were produced via a sol-gel auto-combustion process. A flowchart depicting a procedure of preparing the Tb/Er co-doped ZnO nanopowders (also referred to as ternary metal oxide nanoparticles, and co-doped ZnO powders) is shown in FIG. 1B. Proper amounts of Zn-, Tb-, and Er-based nitrates were weighed in stoichiometric ratios. Each weighed nitrate was dissolved in 30 mL of deionized (DI) water under continuous stirring for about 15 minutes. Then, the different solutions were mixed and stirred at 80° C. until a clear solution was obtained. In the meantime, C₆H₈O₇ was added to the solution to assist with the auto-combustion reaction. Subsequently, the pH was adjusted to 7 by adding dropwise NH₃H₂O. The solution was maintained under stirring and heating until the water was evaporated and a viscous gel was realized. The temperature was further raised to 300° C., and after some time, the gel was self-burnt to give powders. The product obtained was calcined at 500° C. for 4 hours to obtain the co-doped ZnO powders. Pure ZnO nanopowder was synthesized following similar conditions and processes without adding Tb and Er-based nitrates. For each prepared composition, the chemical formula and code of the samples are as follows: ZnO (pure), ZnErTbO-I (Zn_{0.98}Er_{0.01}Tb_{0.01}O), ZnErTbO-II (Zn_{0.94}Er_{0.03}Tb_{0.03}O), and ZnErTbO-III (Zn_{0.90}Er_{0.05}Tb_{0.05}O).

### Example 3: Determination of Anti-Cancerous Activity

Anticancer activities of the prepared samples were performed by a 3-(4,5-Dimethylthiazol-2-yl)-2,5-diphenyltetrazolium bromide (MTT) assay as reported in the previous works [El Rayes, S. M., et al., Convenient Synthesis and Anticancer Activity of Methyl 2-[3-(3-Phenyl-quinoxalin-2-ylsulfanyl)propanamido]alkanoates and N-Alkyl 3-((3-Phenyl-quinoxalin-2-yl)sulfanyl)propanamides, ACS Omega, 2019, 4, 20, 18555-18566, which is incorporated herein by reference in its entirety]. Human cervical cancer (HeLa) and human colorectal carcinoma (HCT-116) cells (provided by ATCC, USA) were utilized to evaluate the anti-cancer performance of the samples. Healthy embryonic kidney (HEK-293) cells were used as a control. First, cells were dispersed in cell culture plates having special Dulbecco modified eagle medium (DMEM) media and incubated in a CO₂ incubator. 5 to 40 μg/mL of prepared samples were added into the culture plates comprising HEK-293, HCT-116, and HeLa cells for 48 hours for subsequent MTT assays [Ansari, M. A. et al., Biofabricated fatty acids-capped silver nanoparticles as potential antibacterial, antifungal, antibiofilm and anticancer agents, Pharmaceuticals, 2021, 14, 2, 139; and Asiri, S. M. et al., Synthesis of chitosan nanoparticles, chitosan-bulk, chitosan nanoparticles conjugated with glutaraldehyde with strong anti-cancer proliferative capabilities, Artif Cells Nanomed Biotechnol., 2018, 46, which are incorporated herein by references in their entireties]. In the control wells, the ZnO nanopowder samples were not included. After that, cells were subjected to MTT (5 mg/mL) for 4 hours, and the media was replaced with dimethyl sulfoxide (DMSO). The plates were analyzed using a Plate Reader (Bio-Tek Instruments) at a wavelength of 570 nm, and optical density (OD) was obtained to determine the cell viability percentage. The results, given in the graphs as mean±standard deviation, are acquired from triplicates and statistically assessed by Prism GraphPad 6.0 Software. After treatment, the cells were stained with 4′,6-diamidino-2-phenylindole (DAPI) stain for 48 h with the prepared samples at a dose of 40 μg/mL to examine the nucleus of HCT-116 cancerous cells. The treatment of cells was performed using paraformaldehyde and then marked with DAPI dye. The blue, fluorescent staining was analyzed using a Zeiss confocal scanning microscope.

### Example 4: Density Functional Theory (DFT) Calculations

Numerical calculations based on first principles within density functional theory (DFT) are employed to demonstrate the influence of Er and Tb dopants on both electronic structure and optical properties of ZnO. The modeling and investigation of the doped systems are conducted using the QuantumATK software [QuantumATK version S-2021.06, Synopsys, which is incorporated herein by reference in its entirety]. Total density of states (TDOS), partial density of states (PDOS), band gap energy (Eg), and parameters describing the optical spectra expose characteristics of the samples.

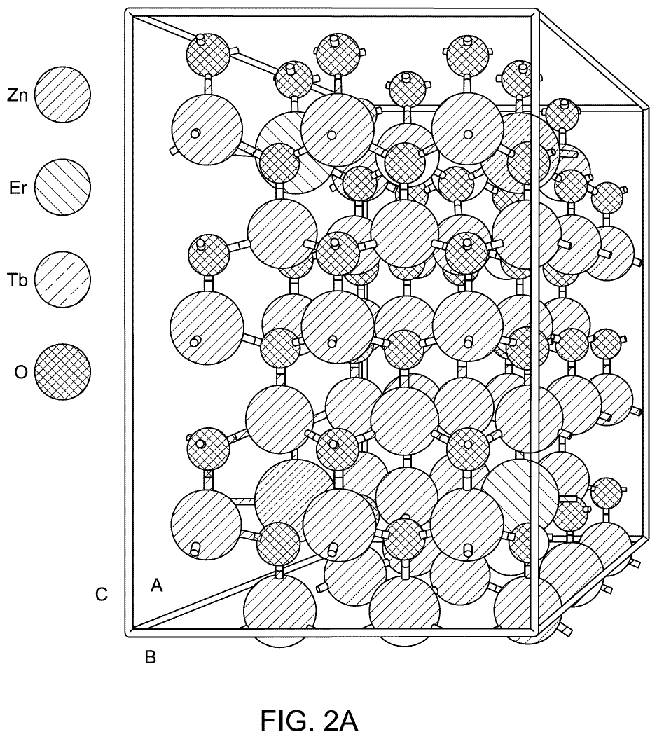

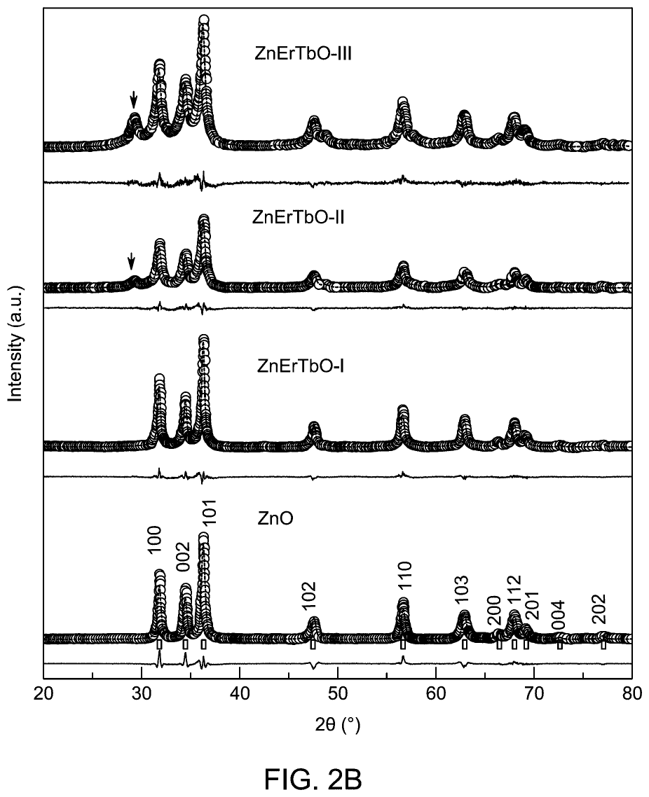

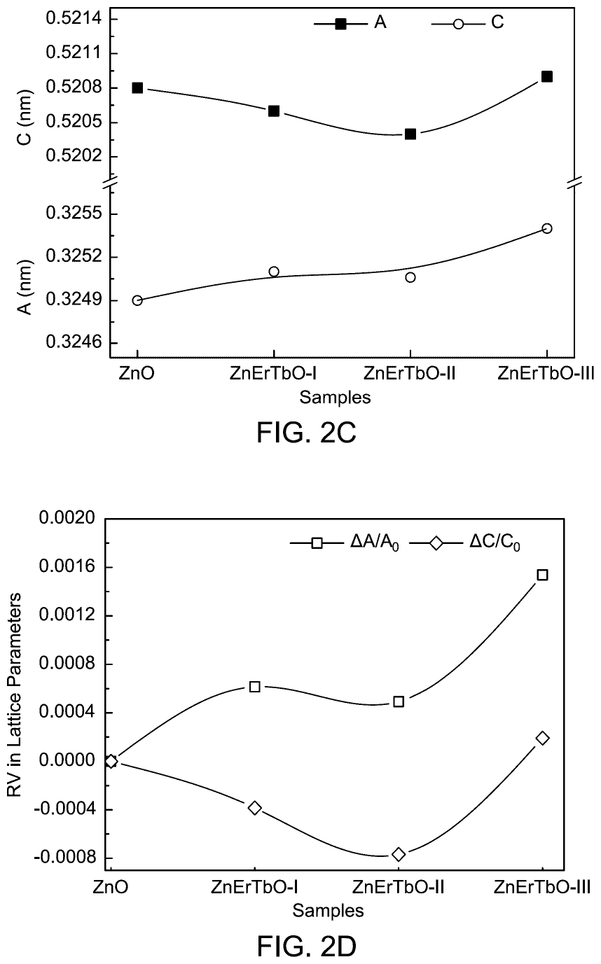

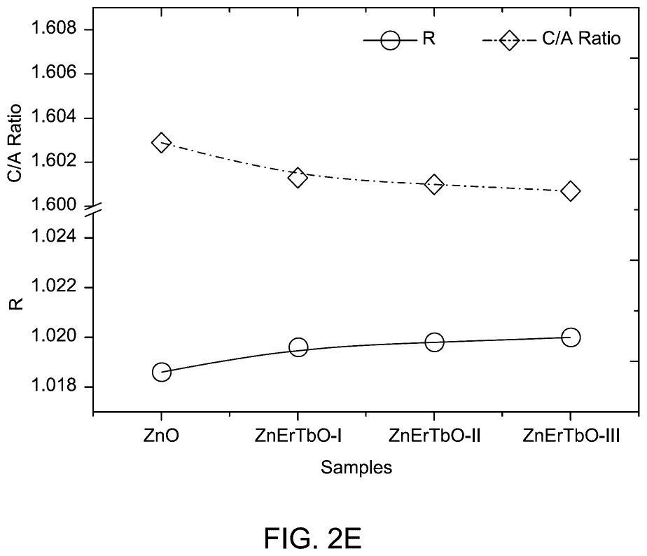

To construct a pure ZnO system, a supercell (3×3×3) consisting of 108 atoms (with bulk lattice parameters A=9.643 Å, B=9.643 Å, C=15.490 Å) is applied. Subsequently, the ZnO system was doped with both Er and Tb to form three different co-doping contents: 1.85% (ZnErTbO-I), 3.70% (ZnErTbO-II), and 5.56% (ZnErTbO-III). FIG. 2A illustrates a representative unit cell that displays the supercell structure of ZnErTbO-II. To optimize (relax) the systems, a 0.01 electron volt (eV)/angstrom (A) tolerance force is utilized. The accompanying calculations employ the generalized gradient approximation (GGA) with the Perdew-Burke-Ernzerhof (PBE) functional (GGA.PBE). The adoption of GGA is justified as it adequately describes the ion distribution in a system. Including the Hubbard (U) term introduces a correction to the approximation. After conducting an initial analysis, this term is assigned a value of 9 eV for the Zn-3d, 6.0 eV for both Er-4f and Tb-4f, and 7.0 eV for the O-2p electrons. The norm-conserving Fritz-Haber-Institute (FHI) pseudopotentials describe the ion cores of atoms. The mesh cutoff energy is selected as 100 Hartree, employing a (6,6,3) k-point mesh within the Monkhorst-Pack scheme. The k-points are increased to (10, 10, 5) for the density of states (DOS) and optical spectra. The double zeta polarized basis sets of local numerical orbitals characterize the electronic structure of valence electrons (Zn: 4s²3d¹⁰, Er: 6s²4f¹², Tb: 6s²4f⁹, and O: 2s²2p⁴).

Crystallographic structure and morphology: The crystalline structure of produced pristine ZnO and Tb/Er co-doped ZnO NPs was investigated via X-ray diffraction (XRD), as shown in FIG. 2B. The phase identification was performed using Match 3! software and the crystal system, space group, and cell parameters were determined via Rietveld refinement by utilizing the FullProf Suite program. In FIG. 2B, the empty dots and solid lines are the observed and calculated profiles, respectively. The line under each spectrum is the difference curve between the calculated and observed profiles. The tick marks are Bragg position reflections of ZnO. The fitting parameters are listed in Table 1. The quality of fitting was judged by computing the parameter S using pattern profile (Rₚ) and weighted profile pattern (R_{wp}) through the formula S=R_{wp}/Rₚ, and by determining the goodness of fit factor ‘χ². Low values of χ² were obtained and the values of the S parameter were close to unity suggesting that the cell parameters resulting from the refinement procedure are relevant. The observed XRD profiles presented in FIG. 2B validate the creation of a wurtzite hexagonal structure (JCPDS card No. 36-1451). No extra phases associated with the erbium element have been detected, signifying that Er³⁺ ions are well incorporated within the ZnO lattice; however, with further increasing the co-dopants content, a small peak around 29.2° matching with the (111) plane of Tb₂O₃ (JCPDS card No. 65-6985) is detected. A small fraction of the Tb³⁺ may occupy the Zn²⁺ sublattices or interstitial sites of the lattice but most of the Tb³⁺ has the tendency to segregate on the ZnO surface in the form of a terbium(III) oxide (Tb₂O₃) secondary phase [Kumar, V. et al., Tunable and white emission from ZnO:Tb³⁺ nanophosphors for solid state lighting applications, Chemical Engineering Journal, 2014, 255, 541-552, which is incorporated herein by reference in its entirety]. Peaks related to an erbium element or erbium oxide (Er₂O₃) were not detected at across diverse doping contents, suggesting that the main Zn²⁺ ions are well replaced by Er³⁺ ions even at x=0.05.

Lattice parameters (a and c) values taken after Rietveld refinement are shown in FIG. 2C. The lattice constants a and c display a slight change, as shown in FIG. 2C, but both the hexagonal crystal system and space group (P6₃mc) remain unchanged, as shown in Table 1. It is common that when doping a ZnO structure with ions having different ionic radii, three possibilities may occur: the dopant ions enter the ZnO structure by replacing substitutional Zn²⁺ sites or they replace the interstitial Zn²⁺ sites or they appear as secondary phases that accumulate on the surface of ZnO network and generate defects [Sharma, A. et al., A study of structural parameters and photoluminescence of Tb doped ZnO nanoparticles, Materials Today: Proceedings, 2020, 26, 58-63, which is incorporated herein by reference in its entirety]. According to Vegard's law, to accommodate dopants within the host lattice, the host lattice parameters either increase or decrease depending on the ionic radius of the dopants (either larger or smaller). In the case of the present disclosure, the cell was doped with erbium and terbium ions which have larger ionic radii compared to that of Zn²⁺, thus an expansion of the unit cell volume of ZnO may occur. To further illustrate the influence of co-doping on the structural properties of ZnO, the relative variation (RV) in lattice parameters Δa/a₀ and Δ_{c}/c₀ (where a₀ and c₀ refer to the lattice parameters of pristine ZnO sample) are plotted in FIG. 2D. Non-monotonic variations in lattice parameters with co-doping amounts were observed for the as-prepared samples. Both lattice parameters “a” and “c” showed a RV increase in the ZnErTbO-III sample, indicating an expansion of the unit cell volume caused by the amounts of Er and Tb elements in the sample. This effect may be attributed to the crystal distortion produced by the bigger ionic radii of co-dopants (r_{Tb+}=0.92 Å and r_{Er3+}=0.89 Å) compared to the ionic radius of the host (r_{Zn3+}=0.74 Å) [Hastir, A. et al., Temperature dependent selective and sensitive terbium doped ZnO nanostructures, Sensors and Actuators B: Chemical, 2016, 231, 110-119; and Achehboune, M. et al., A DFT study on the electronic structure, magnetic and optical properties of Er doped ZnO: Effect of Er concentrations and native defects, Computational Condensed Matter, 2022, 31, which are incorporated herein by references in their entireties]. The degree of distortion (R=(2a√(⅔))/c) was determined and their values for different products are plotted in FIG. 2E. R increases as the contents of Er and Tb increase, supporting the occurrence of distortion in the ZnO matrix caused by the co-doping effect. A comparable result was obtained in a Tb-doped ZnO sample synthesized via a sol-gel method where a rise in the lattice parameters was observed when the Tb mole fraction (x) exceeded 0.04. Below this value, a shrinkage in unit cell volume was attributed to the behavior to the accumulation of Tb ions on the surface of ZnO, creating defects and hydrostatic strain, which alter the surface of the host material for the initial concentrations of Tb dopants [Sharma, A. et al., A study of structural parameters and photoluminescence of Tb doped ZnO nanoparticles, Mater. Today: Proc., 2020, 58-63, which is incorporated herein by reference in its entirety]. The variation of the c/a ratio for different samples is depicted in FIG. 2E. The values are 1.6028, 1.6013, 1.6010, and 1.6007 for ZnO, ZnErTbO-I, ZnErTbO-II, and ZnErTbO-III, respectively, which are coherent with that of the common value (c/a=1.6) [Yang, X. et al., Volume-matched ferroelectric and piezoelectric ZnO/MgO superlattice, Journal of Alloys and Compounds, 2021, 876, 160167, which is incorporated herein by reference in its entirety].

TABLE 1
Structure, space group, and fitting parameters

| Samples | | ZnO | ZnErTbO-I | ZnErTbO-II | ZnErTbO-III |
|---|---|---|---|---|---|
| Structure | | Hexagonal | Hexagonal | Hexagonal | Hexagonal |
| Space group | | P6₃mc | P6₃mc | P6₃mc | P6₃mc |
| Fitting parameters | χ² | 3.6 | 2.2 | 1.5 | 1.5 |
| | S = R_{wp}/Rₚ | 1.1 | 1.1 | 1.09 | 1.1 |

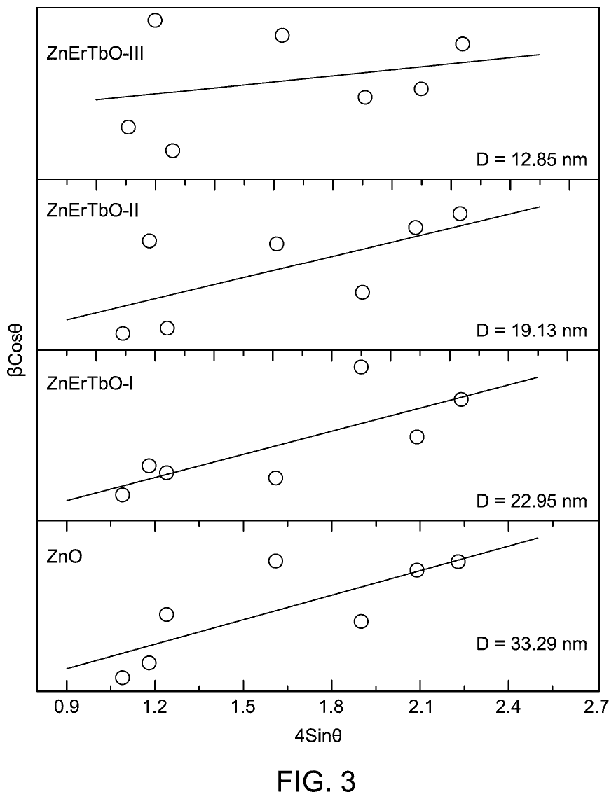

Another apparent effect is the widening of peaks for co-doped samples compared to pure ZnO. This may result from the joint contribution of strains in the matrix owing to lattice distortion and a decrease in crystallite size. Williamson-Hall (WH) plots were used to determine crystallite size (from the Y-intercepts) and strain (from the slopes) [Al Boukhari, J. et al., Structural analysis and dielectric investigations of pure and rare earth elements (Y and Gd) doped NiO nanoparticles, Journal of Alloys and Compounds, 2020, 820, 153381, which is incorporated herein by reference in its entirety]. FIG. 3 displays the plots of βₕₖₗ cos θ versus 4 sin θ for ZnO, ZnErTbO-I, ZnErTbO-II, and ZnErTbO-III. The crystallites size of all prepared samples is in the order of a nanometer and decreases with Er and Tb co-doping in different amounts. The linear plots of all samples show a positive slope. The strain varies with Er and Tb ions co-doping, and its value lies between 0.92 and 1.44(×10⁻³). The reduction in the crystallite size is attributed to the distortion in the ZnO lattice by Er and Tb co-dopant ions, which restrains the rate growth of ZnO grains [Manikandan, A. et al., Rare earth element (REE) lanthanum doped zinc oxide (La:ZnO) nanomaterials: synthesis structural optical and antibacterial studies, Journal of Alloys and Compounds, 2017, 723, 1155-1161, which is incorporated herein by reference in its entirety]. The evolution of crystallite size is correlated with the relative interrelationship between the nucleation and growth steps [Yadav, S. P. et al., Distribution of cations in Co₁₋ₓMnₓFe₂O₄ using XRD, magnetization and Mössbauer spectroscopy, Journal of Alloys and Compounds, 2015, 646, 550-556, which is incorporated herein by reference in its entirety], and it is influenced by the preparation procedure, temperature, and nature of dopants. Crystalline size of zinc oxide decreases due to doping with RE elements. A monotonic diminution in crystallite size was achieved by increasing the content of La³⁺ into ZnO prepared via co-precipitation. A sharp decrease in crystallite size from 73 nm for pristine hydrothermally prepared ZnO nanorods to 56 nm for 1 (weight percent) wt. % Er-doped ZnO sample was observed [Achehboune, M. et al., Optimization of the luminescence and structural properties of Er-doped ZnO nanostructures: effect of dopant concentration and excitation wavelength, Journal of Luminescence, 2022, 246, 118843, which is incorporated herein by reference in its entirety]. A reduction in crystallite size to 21 nm for Tb and Eu doped-ZnO NPs synthesized via an auto-combustion process compared to undoped ZnO (32 nm) has been observed [Ahmad, I. et al., Rare earth co-doped ZnO photocatalysts: solution combustion synthesis and environmental applications, Separ. Purif. Technol., 2020, 237, 116328, which is incorporated herein by reference in its entirety]. A reduction in the values of crystallite size following co-doping Tb and Yb has also been observed in sprayed ZnO thin films [El Hat, A. et al., Growth and characterization of (Tb, Yb) Co-doping sprayed ZnO thin films, Crystals, 2020, 10, 3, 169, which is incorporated herein by reference in its entirety].

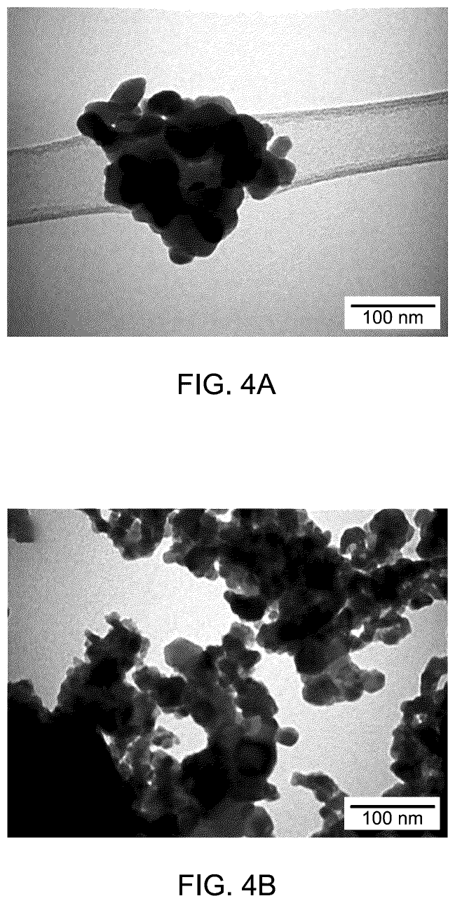

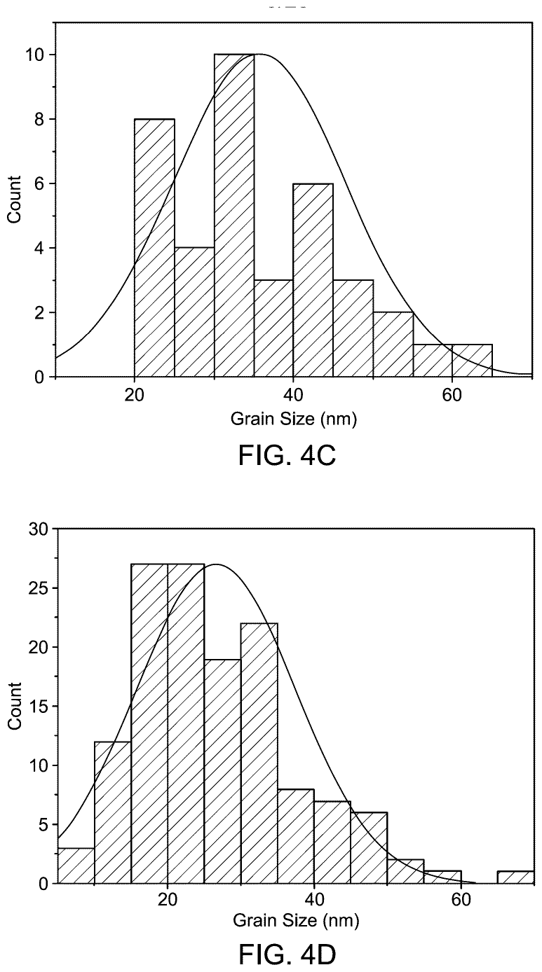

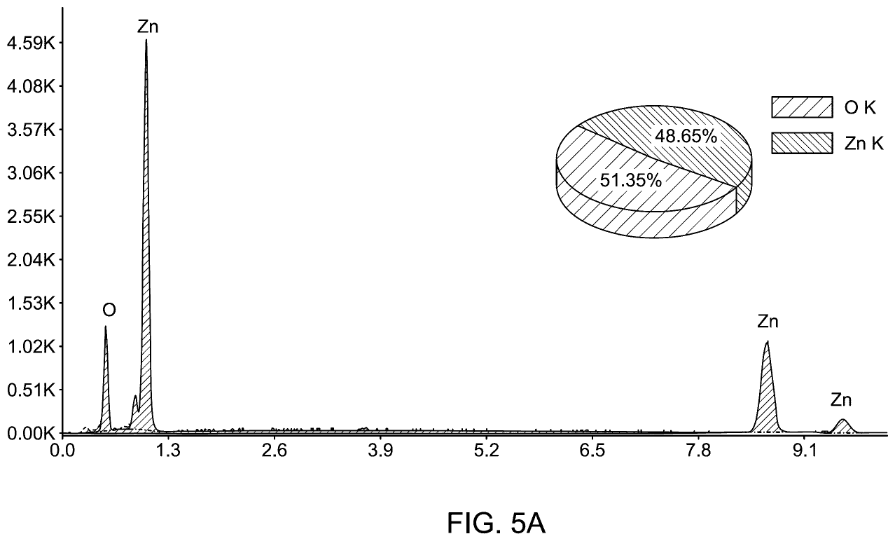

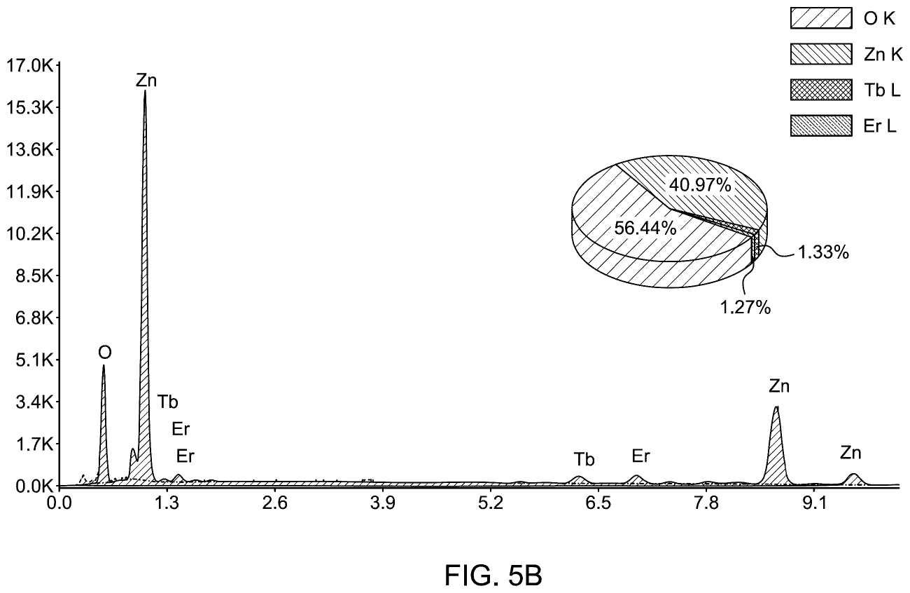

Transmission electron microscopy (TEM) images are presented in FIGS. 4A-4B for pure ZnO and ZnErTbO-II, respectively. The average particle size is 35.67 nm and 26.58 nm for pure ZnO and ZnErTbO-II, respectively. The particle size decreased upon Tb and Er co-doping, which is in accordance with the variation of crystallite size as obtained by XRD analysis and shown in FIG. 4C (ZnO) and FIG. 4D (ZnErTbO-II). Elemental composition and chemical purity of undoped and co-doped ZnO NPs were studied by energy dispersive X-ray analysis (EDAX) spectroscopy, as shown in FIGS. 5A-5B. The EDX spectrum for undoped ZnO is presented in FIG. 5A and confirmed the occurrence of zinc and oxygen elements with atomic percentages of 48.65% and 51.35%, respectively. For the co-doped sample, as shown in FIG. 5B, peaks associated with Tb and Er elements have appeared. No extra peaks related to other elements were detected, confirming the purity and the successful formation of the desired compositions.

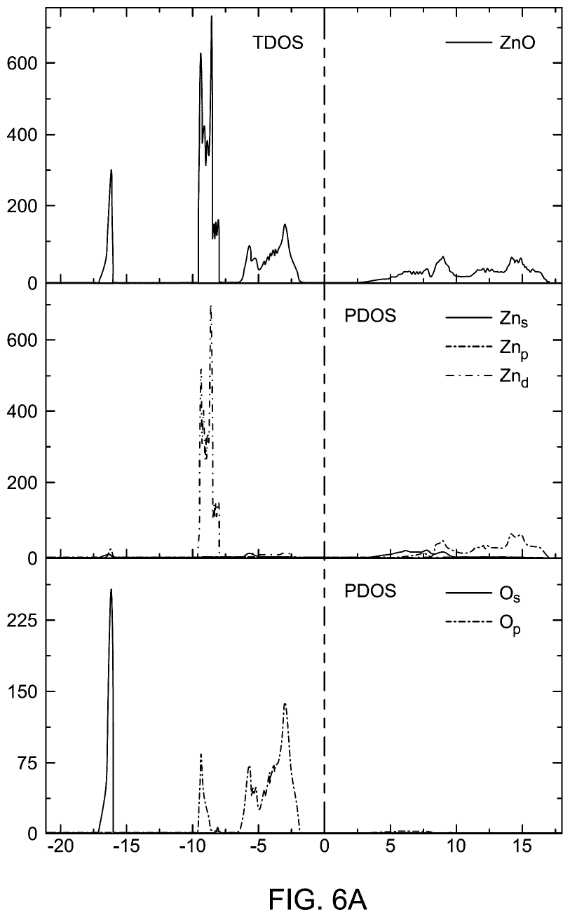

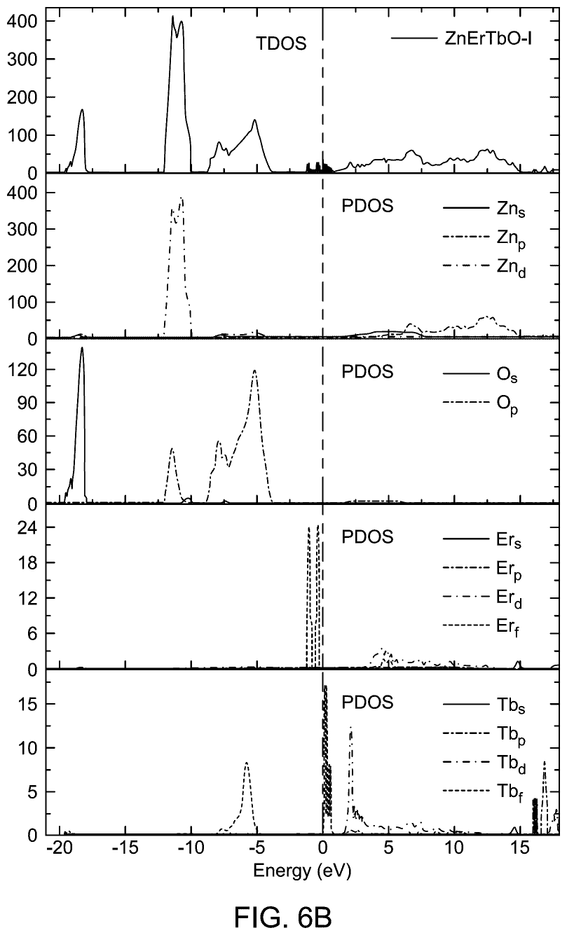

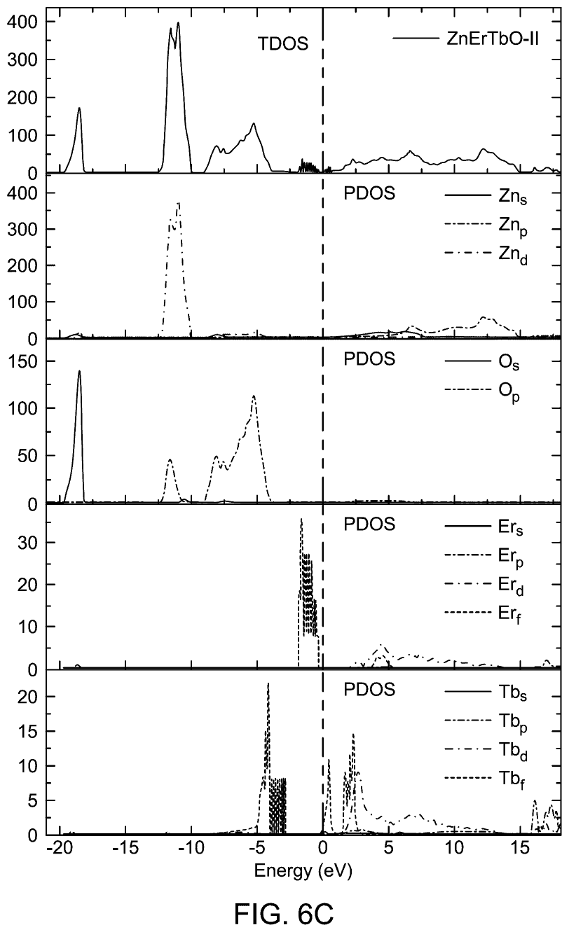

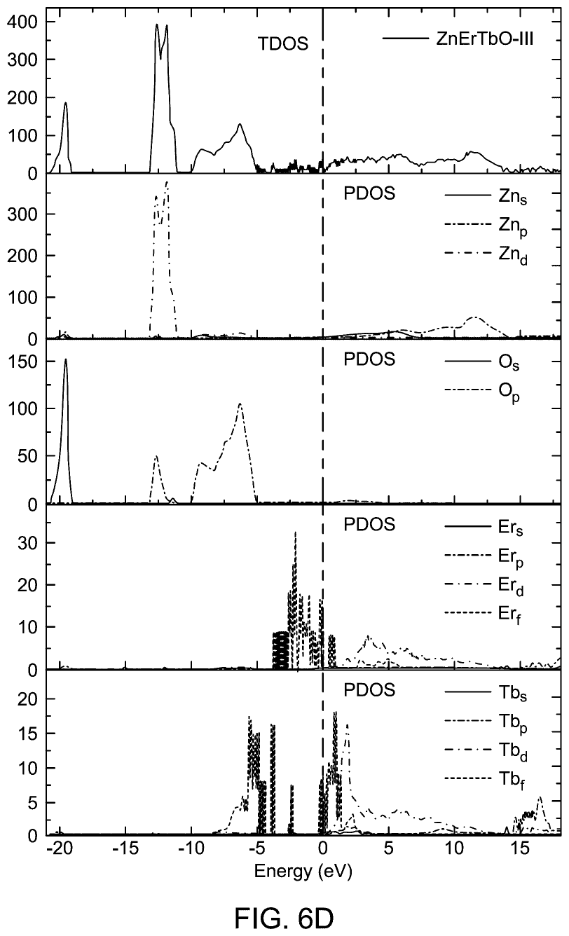

Theoretical study: Partial density of states (PDOS) and total density of states (TDOS) spectra are presented in FIGS. 6A-6D and provide insights into electronic structure features of undoped and co-doped ZnO NPs. In the spectra, the Fermi energy (EF) is taken as the reference point. By comparing TDOS spectra, it may be observed that the EF shifts towards the conduction band (CB) when co-doping is introduced, as shown in FIGS. 6B-6D. Moving of EF into the conduction band upon doping ZnO materials with rare earth atoms has been observed before [Sharma, A. et al., A study of structural parameters and photoluminescence of Tb doped ZnO nanoparticles, Materials Today: Proceedings, 2020, 26, 58-63; Achehboune, M. et al., A DFT study on the electronic structure, magnetic and optical properties of Er-doped ZnO: Effect of Er concentration and native defects, Computational Condensed Matter, 2022, 31, e00627; and Honglin, L. et al., Experimental and first-principles studies of structural and optical properties of rare earth (RE=La, Er, Nd) doped ZnO, Journal of Alloys and Compounds, 2014, 617, 102-107, which are incorporated herein by reference in their entireties]. This shift towards the conduction band indicates the presence of n-type conductivity, which occurs as the Er and Tb rare earth atoms possess more valence electrons compared to those forming the host structure. The present disclosure further confirms the findings through the co-doping of rare earth Er and Tb atoms in ZnO. The Eg of undoped ZnO is determined as 3.45 eV. In the case of ZnErTbO-I, ZnErTbO-II, and ZnErTbO-III, the respective Eg values are 2.89 eV, 2.26 eV, and 1.97 eV. Consequently, the Tb/Er co-doping leads to a narrower band gap, due to the formation of new levels of energy. Adjusting the band gap with rare earth atoms is an effective way for envisaged applications in optoelectronics. The PDOS plot in FIG. 6A indicates that the valence band (VB) of undoped ZnO primarily arises from the Zn-3d and O-2p orbitals within the energy range of −9.5 eV to −2.0 eV, suggesting a hybridization between Zn and O atoms. Additionally, the O-2s orbitals contribution to the TDOS is observed around −17 eV, as shown in FIG. 6A. For doped ZnO NPs, the d orbitals of both Er and Tb mainly contribute to the conduction band. While the Er-4f electrons chiefly impact the valence band only, the Tb-4f orbitals influence both bands, as shown in FIGS. 6B-6D in both pure and doped ZnO systems, the p orbitals of Zn atoms have an appreciable effect in the conduction band where d orbitals of both dopants and Tb-4f electrons play a role as well, as shown in FIGS. 5A-5B. The PDOS plot of ZnErTbO-I demonstrates that both Er-4f and Tb-4f orbitals are responsible for the occupied states at the EF, as shown in FIG. 6B. With the insertion of further Er and Tb dopants (yielding the ZnErTbO-II and ZnErTbO-III structures), the energy states undergo shifts, as illustrated in FIG. 6C and FIG. 6D. The presence of impurity states, resulting from the dopants, around the Er reveals the photoelectric and optoelectronic properties of semiconducting nanoparticles. When ZnO nanomaterials are doped with RE atoms like Tb and Er, they may be utilized to advance optoelectronic devices.

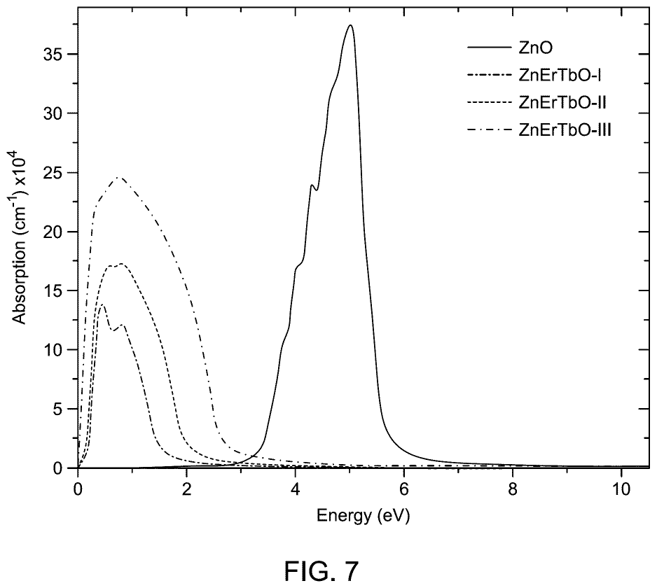

First-principles calculations have been used to investigate nanomaterials' optical characteristics. In FIGS. 7-11, relevant parameters that unveil the optical properties are presented. An absorption coefficient, extinction coefficient, reflectivity, refractive index, dielectric constant, and energy loss function are presented. The peak observed in pure ZnO experiences a shift toward lower energies (a redshift) upon doping, as depicted in FIGS. 7-11. This shift due to doping is consistent with Sm- and Tb-doped ZnO nanoparticles [Wang, J. et al., A GGA+U study of electronic structure and the optical properties of different concentrations Tb doped ZnO, Phys. B Condens. Matter, 2020, 576, 411720, which is incorporated herein by reference in its entirety]. The shift may be attributed to the interaction between the dopants and host Zn atoms. FIG. 7 illustrates the absorption coefficient, showing that the absorption edge of pure ZnO is roughly 3.4 eV. It implies the Eg of pure ZnO, which exhibits a well-defined conspicuous absorption peak (at 5 eV) in the ultraviolet region. The absorption of pure ZnO sharply declines and disappears roughly below 3 e V and above 8 eV. A redshift in the absorption peaks resulted after introducing Er and Tb atoms as dopants to ZnO. It suggests a narrower band or a downward shift of the conduction band edge to lower energies. As soon as dopants are introduced, upon a rapid drop, the absorption peak is boosted with dopant content, as shown in FIG. 7. At 1 eV, the ZnErTbO-III exhibits the greatest absorption peak, which is still less than that of pure ZnO.

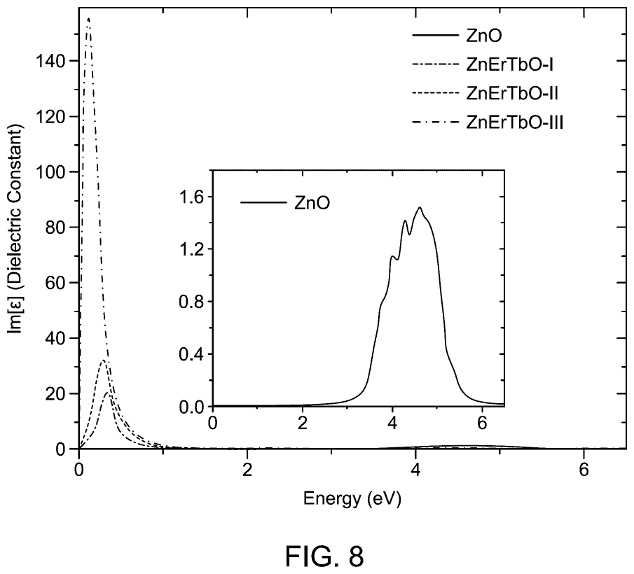

The imaginary part of the dielectric constant, depicted in FIG. 8, is useful for analyzing optical properties of nanoparticles. It signifies energy needed to transition from the valence band (VB) to the conduction band (CB). It is observed to be much weaker (a minute peak near 4.3 eV) for the pure ZnO system (see FIG. 8 and the inset therein). In contrast, the doped ZnO nanoparticles exhibit enhanced sharp peaks that shift to lower energies within the interval of 0.2 to 0.5 eV, as shown in FIG. 8. Furthermore, the peak intensity improves with the dopant concentration, with the utmost intensity monitored for the ZnErTbO-III system. The strength of the peak at a specific energy, in the imaginary part of the dielectric spectrum, is associated with the absorption frequency range.

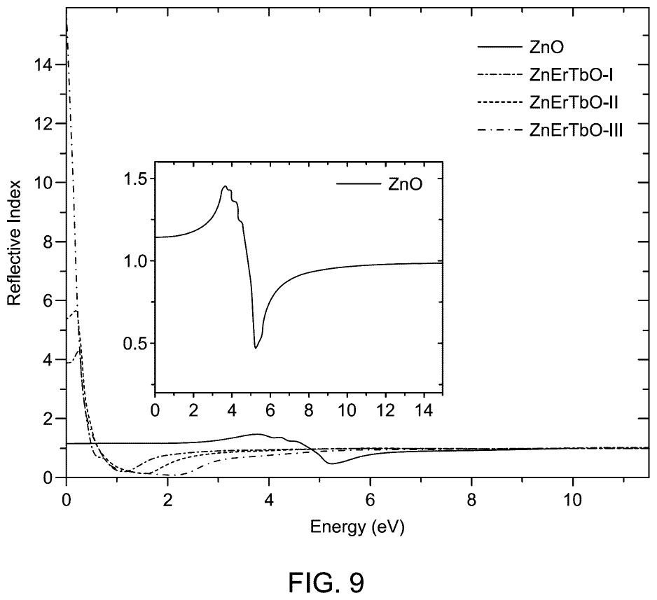

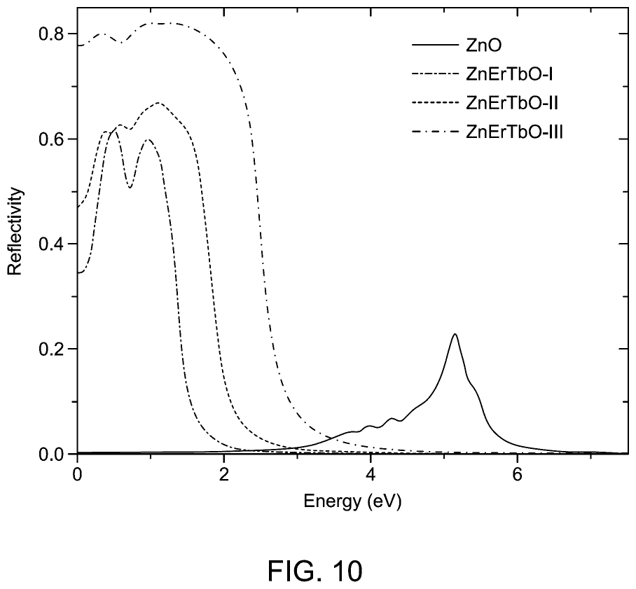

FIG. 9 displays the refractive index presenting a subtle peak at 4 eV for pure ZnO, followed by a dip around 5 eV. Co-doping leads to an improvement in the refractive index along with a redshift. The minimum refractive index describing the doped ZnO nanoparticles is observed in the energy interval from 1.6 eV to 2.2 eV. The reflectivity shown in FIG. 10 demonstrates that the peak, representing the pure ZnO in the ultraviolet region, shifts towards the infrared range (ranging from 0.5 eV to 2.2 eV) when doped by Er and Tb atoms.

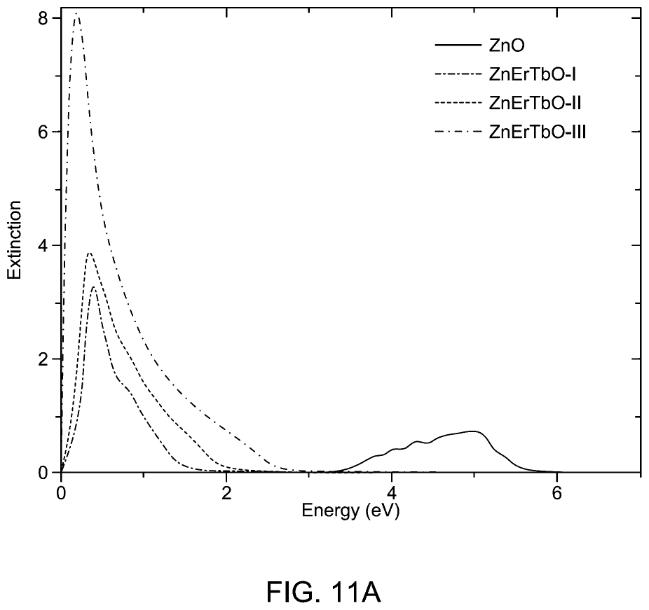

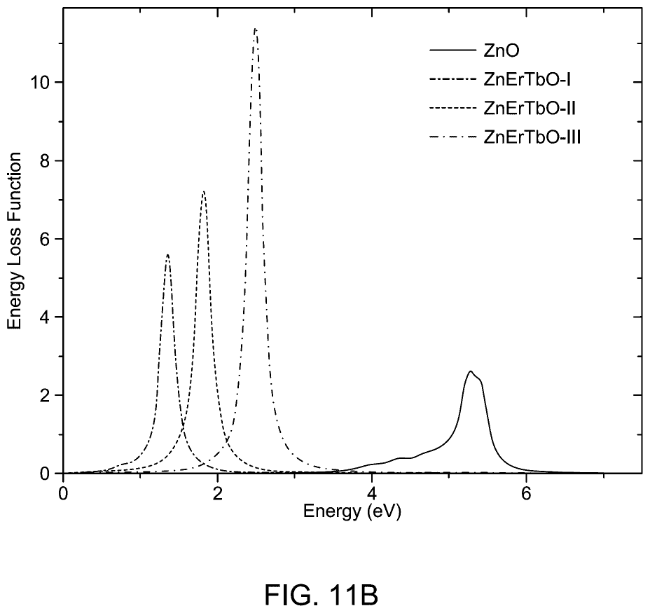

The extinction and energy loss functions are displayed respectively in FIGS. 11A-11B. The extinction spectrum of pure ZnO exhibits a corresponding peak in the ultraviolet region at 5 eV. The doped ZnO nanoparticles demonstrate stronger peaks in the infrared region (near 0.4 eV), as shown in FIG. 11A, where the extinction of ZnErTbO-III is the greatest. The energy loss within the medium may be characterized by the energy loss function, as shown in FIG. 11B, displaying a peak for the pure ZnO positioned at 5.2 eV. Upon doping, the accompanying peaks are shifted to the lower energies in the energy interval of 1.3 to 2.6 eV. ZnErTbO-III exhibited the highest intensity of the energy loss function. The findings through the optical spectra suggest that an appropriate Er and Tb content enhances optical characteristics of ZnO-based nanoparticles.

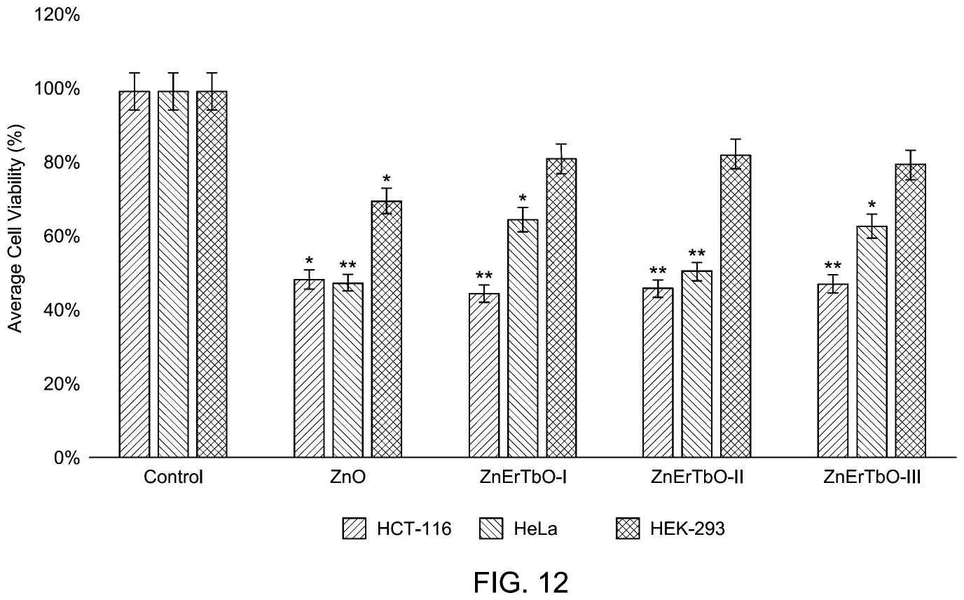

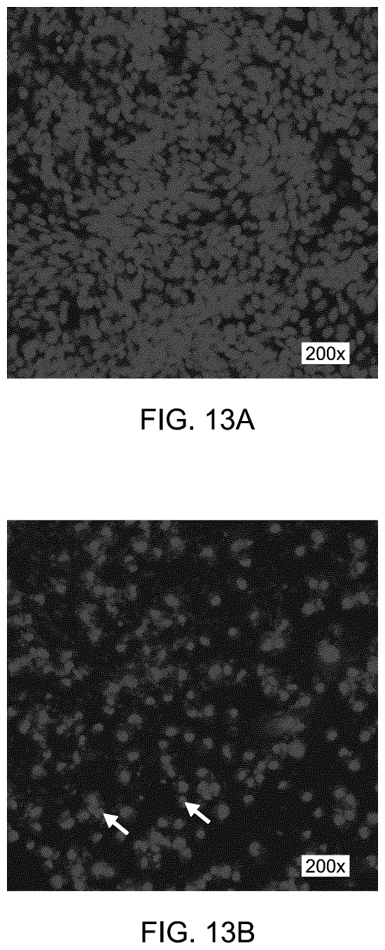

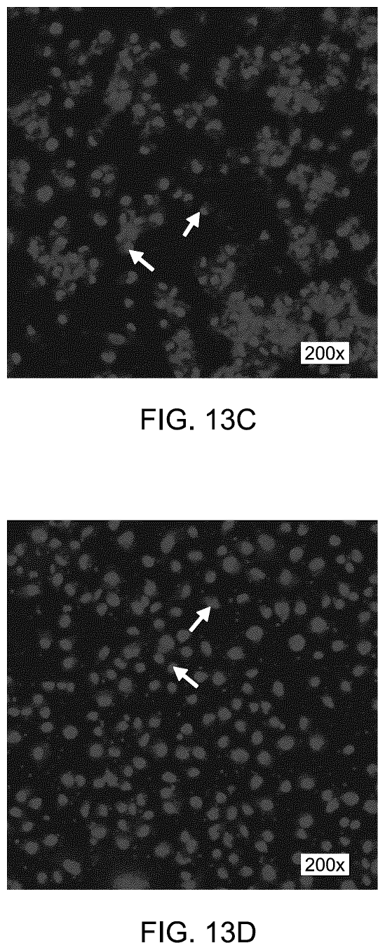

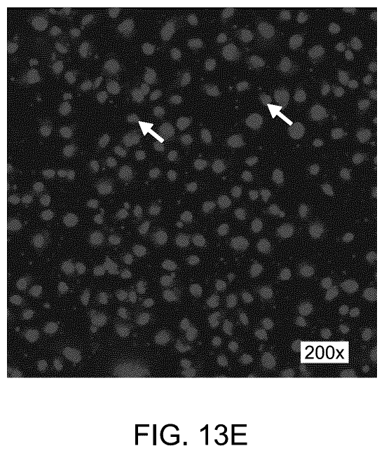

Anti-cancer in-vitro analysis: Nanotechnology has effectively contributed to the discovery and development of innovative methodologies for treating cancer. Due to their physical characteristics, metal oxides are good options for delivery of large and small drug biomolecules, where the most distinct metal oxide is ZnO NPs. Several studies reported the cytotoxicity of undoped and doped ZnO NPs against diverse types of cells [Rama Narsimha Reddy, A. and Srividya, L., Evaluation of In Vitro cytotoxicity of zinc oxide (ZnO) nanoparticles using human cell lines, Journal of Toxicology and Risk Assessment, 2018, 4, 1; and Hamidian, K. et al., Co-doped nanowires: synthesis, photocatalytic performance, and cytotoxic activity against human brain glioblastoma cells, Results in Chemistry, 2023, 5, 100734, which is incorporated herein by references in their entireties]. FIG. 12 shows cell viability assay results for ZnO, ZnErTbO-I, ZnErTbO-II, and ZnErTbO-III. A reduction in the cancerous cells (HCT-116 and HeLa) was noticed after 48 hours of treatment with the synthesized samples. The treatment of undoped ZnO showed 48.63% decrease in the HCT-116 cells, and the treatment of ZnErTbO-I, ZnErTbO-II, and ZnErTbO-III caused a 44.83%, 46.24% and 47.26% decrease in the HCT-116, respectively. When the HeLa cells were treated, the undoped ZnO showed a 47.639% decrease in the HeLa cells, and treatments with ZnErTbO-I, ZnErTbO-II, and ZnErTbO-III caused a 65.04%, 50.77% and 63.41% decrease in the HeLa cells, respectively. Treatment of the synthesized samples was also assessed on normal healthy HEK-293 cells. The treatment of undoped ZnO showed a 69.95% decrease in the HEK-293 cells, and treatments with ZnErTbO-I, ZnErTbO-II, and ZnErTbO-III caused an 81.68%, 82.89% and 80.04% decrease in the HEK-293 cells, respectively. The impact of pure and Tb/Er co-doped ZnO caused more cytotoxicity on the HeLa and HCT-116 cells than HEK-293 cells, as shown in FIG. 12. The produced samples of the present disclosure show strong cytotoxic effects towards HCT-116 and HeLa cells. To better understand the reasons for the cancer cell death, HCT-116 cells were selected because they showed better inhibitory effects compared to HeLa cells. The selected cells were treated with undoped ZnO and Tb/Er co-doped ZnO nanoparticles for 48 hours and labeled with blue-fluorescent DNA stain DAPI to scrutinize the cancer cell DNA after the treatment. DAPI is a blue fluorescent stain that binds strongly to A-T-rich regions in DNA. It shows ~20-fold enhancement of fluorescence upon binding to AT regions of double stranded DNA (dsDNA). Due to its high affinity for DNA, it is also commonly applied for counting cells and measuring apoptosis [Wallberg, F. et al., Analysis of apoptosis and necroptosis by fluorescence-activated cell sorting, Cold Spring Harbor Protocols, 2016, 4; and Yoo, E. S. et al., Antitumor and apoptosis-inducing effects of piperine on human melanoma cells, Anticancer Res., 2019, 39, 4, 1883-1892, which are incorporated herein by references in their entireties]. Treatment with the prepared ZnO, ZnErTbO-I, ZnErTbO-II, and ZnErTbO-III samples showed high cancer cell deaths, as shown in FIGS. 13A-13E. The control group cells (not treated with the prepared nanoparticles) appeared healthy without any morphological changes, as shown in FIG. 13A; however, treatment with ZnO, ZnErTbO-I, ZnErTbO-II, and ZnErTbO-III caused chromatin condensation and formation of apoptotic bodies in the HCT-116 cells, as shown in FIG. 13B-13E.

The present disclosure reports crystallographic structures and morphologies for ZnO co-doped with Er and Tb elements. The impact of co-doping on the electronic structure and optical features was examined using DFT calculations. Experimental analysis done via XRD, EDX, and TEM displayed efficient development of nanosized compositions. The crystallite size of prepared samples decreased and the degree of distortion increased with increasing the co-dopant amounts inside the host ZnO system. The energy band gap of undoped (pure) ZnO was determined to be 3.45 eV, which is reduced with co-doping Er and Tb ions. The absorption edge of pure ZnO, has a salient absorption peak at 5 eV in the ultraviolet region. Er and Tb co-doping induced an improved refractive index. The narrower band gap, due to the Er and Tb dopants, results in a red shift of absorption peaks, among which the highest one is for ZnErTbO-III at 1 eV. When the ZnO is doped with Er and Tb, for the imaginary part of the dielectric constant, enhanced sharp peaks that shift to lower energies (within the range of 0.2 to 0.5 eV) are observed. Cell viability assay findings showed a diminution in HeLa and HCT-116 cancerous cells after 48 hours of treatment with the samples. In comparison to the undoped sample, the co-doped ZnO NPs showed better anti-cancer activity against HCT-116 cells. It was established that the treatment of ZnErTbO-I, ZnErTbO-II, and ZnErTbO-III caused a 44.83%, 46.24%, and 47.26% decline in the HCT-116 cells viability, respectively. Treatment with co-doped ZnO samples produced chromatin condensation and formation of apoptotic bodies in HCT-116 cells, which indicates that prepared Er/Tb co-doped ZnO NPs triggered programmed cell death in HCT-116 cells. The findings of the present disclosure show that Tb/Er co-doped ZnO may be a versatile candidate in various fields, as it may be considered new optoelectronic device applications and may be recommended as a potential for cancer treatment.

Numerous modifications and variations of the present disclosure are possible in light of the above teachings. It is, therefore, to be understood that within the scope of the appended claims, the disclosure may be practiced otherwise than as specifically described herein.

1: A method of treating cancer cells, comprising:
contacting cancer cells with ternary metal oxide nanoparticles to induce cell death of the cancer cells,
wherein the ternary metal oxide nanoparticles comprise a zinc oxide matrix doped with terbium and erbium,
wherein the terbium is present in an amount of 0.5 to 10 atomic percent (at. %); and
the erbium is present in an amount of 0.5 to 10 at. %,
wherein the atomic percent is based on a total atom count of zinc, terbium, and erbium,
wherein the erbium replaces zinc within the zinc oxide matrix,
wherein the ternary metal oxide nanoparticles have an average particle size of 22 to 32 nm.

2: The method of claim 1, wherein the terbium is on a surface of the zinc oxide matrix.

3: The method of claim 1, wherein the ternary metal oxide nanoparticles do not contain an erbium oxide phase.

4: The method of claim 1, wherein the ternary metal oxide nanoparticles comprise a terbium oxide phase.

5: The method of claim 1, wherein the ternary metal oxide nanoparticles have a lattice parameter (a) of 0.32 to 0.33 nm.

6: The method of claim 1, wherein the ternary metal oxide nanoparticles have a lattice parameter (c) of 0.51 to 0.53 nm.

7: The method of claim 1, wherein the ternary metal oxide nanoparticles have a band gap energy of 1.5 to 3 eV.

8: The method of claim 1, wherein the ternary metal oxide nanoparticles have a crystallite size of 10 to 25 nm.

9: The method of claim 1, wherein the ternary metal oxide nanoparticles comprise oxygen in an amount of 50 to 60 at. %, zinc in an amount of 35 to 45 at. %, terbium in an amount of 0.5 to 5 at. %, and erbium in an amount of 0.5 to 5 at. % based on a total atom count of the oxygen, zinc, terbium, and erbium.

10: The method of claim 1, wherein the ternary metal oxide nanoparticles have a peak in an extinction spectrum at 0.1 to 1 eV corresponding to an infrared region.

11: The method of claim 1, wherein the ternary metal oxide nanoparticles have a peak in an energy loss spectrum at 1 to 3 eV.

12: The method of claim 1, wherein the contacting occurs for 36 to 60 hours.

13: The method of claim 1, wherein the cancer cells are HCT-116 cells, and the HCT-116 cells have an average cell viability of 44 to 48% after 45 to 50 hours.

14: The method of claim 1, wherein the cancer cells are HeLa cells, and the HeLa cells have an average cell viability of 50 to 66% after 45 to 50 hours.

15: The method of claim 1, wherein a concentration of the ternary metal oxide nanoparticles is 30 to 50 μg/mL.

16: The method of claim 1, wherein apoptotic bodies are formed to induce cell death of the cancer cells.

17: The method of claim 1, wherein chromatin condensation occurs to induce cell death of the cancer cells.

18: The method of claim 1, wherein the ternary metal oxide nanoparticles are made by a process comprising:
dissolving a zinc salt in a polar protic solvent to form a zinc solution;
dissolving a terbium salt in a polar protic solvent to form a terbium solution;
dissolving an erbium salt in a polar protic solvent to form an erbium solution;
mixing and heating the zinc solution, the terbium solution, and the erbium solution to form a reaction solution;
adding an acid to the reaction solution;
adjusting the pH of the reaction solution to 6 to 8 with a base;
heating the reaction solution to 200 to 400° C. to form a powder; and
calcinating the powder for 3 to 5 hours at 400 to 600° C. to form the ternary metal oxide nanoparticles.

19: The method of claim 1, wherein the ternary metal oxide nanoparticles comprise terbium in an amount of 0.9 to 5.5 at. % based on a total atom count of zinc, terbium, and erbium.

20: The method of claim 1, wherein the ternary metal oxide nanoparticles comprise erbium in an amount of 0.9 to 5.5 at. % based on a total atom count of zinc, terbium, and erbium.
